SEQUENCE LISTING (36 sequences)

SEQ ID NO: 1 - protein, 313 aa, Jilunia laotingensis
MKTIKLKYTDFWDHFDPKNNFITNVLSKKYHIEFSDTPDYLIFSIFGYEHLKFQNCIKILYSGENITPDFNLCDYAIGFDHITFGDRYMRLPLYVSYDIERLAKPKAIDPEKVLNRKFCSFVVSNGVSPERNRFFKLLSEYKRVDSGGRFMNNIGGPVPDKEAFIKDYKFNIAFENSIYDGYTTEKLLEPMLMDSLPIYWGNKLVGLDFNPQSFINATDFPSLEAVVEYVVKLDTNDEDYLSKLSIPWLNKNNYLNWESQFYDFFDNIFNKPKSEARYLTDYGRVRIYRQRLNLFSTIDVIKRKLDDLRNRFR

SEQ ID NO: 2 - protein, 323 aa, unidentified
MNNKIKINFVDFWGDFNKCDNFITRALSKKYEVAISDNPDYLFFGTFGYRHLNYNCVKIMFIGENIAPDFNLCDYALGFDFIQFGDRYMRLPLYCTYESFASLAQTRLPSDEALIDRKFCSMVVSNNRHASPHRERFFKLLSEYKQVDSGGRLWNNVGGPVANKLEFVSQYKFNIAFENSAVHGYTTEKIMEPMAVNTLPLYWGNPLVCKDFNAASFVNVNDFASMEDAVQHIVDLDNDDSKYLQMIKAPKVLDENIINWEERLLDFLSNIIEKPIEEARYLAPFGVQKLYREELLAYDILSRRLKVKKIVAAYDKLRKKIRG

SEQ ID NO: 3 - protein, 320 aa, unidentified
MRDVTVAFVDFWPAFDDKNNFILDVLRQKYNVKTVAASDCPDILFYSYFGIENLRHTEPVRVYFTGENDVPDFNLCDYAISFHHLSFANRHLRLPLYVIYSCFNDLRNGRILQCEDAENRDFCSMVVSNVKNSDMARLDFFNLLSGYKQVASGGRFANNVGGCVTDKLSFISGYKFNIAFENSRVEGYVTEKIIEAFSSGTLPIYWGDPLVANELNDKAFIDVSKFDSLDAAVKFVAALDNNREQYLEILNQPPLLNNPFLNWEEMLLDFLTPIVNELKKYRVDTGAIGTAYRHKMSREQIYNIVTLKKPFELFKHLFHS

SEQ ID NO: 4 - protein, 347 aa, Cryptobacteroides onthequi
MPKKHIKILFVDFWDNFSPTDNLFVNLLSDAYDIEVSETPDYLFYSVFGQKHLKYRDCVRIFYTGENQSPDFNVCDYAVGFDHITFMDRYHRFPLYVQFAGDYDDMMSKHARTDGELLAGRDGFCSFVYSNNNASPERNRFYDLLCTYKPVASGGRFRNNVGGPVADKKEFQSHYRFSVAFENTSVPGYATEKLVQAFAAGTVPVYWGDPLIDRTFNSKAFINCHDYDSFEDVLAAVRRIDQDESLYLAMLREPALIDMNDTRERRLEELRSFLCHILDQDPVSARRYSREYWALRYYDNLEVRETVYRRSLRGIAESFYKKYFWRWRRSSRLMWKIDRFLKRLIGR

SEQ ID NO: 5 - protein, 321 aa, Bacteroides gallinaceum
MKSLKIKFVDFWPGFNPNDNFITNALKGYEIVITDTPDYLFFSIFGYSHLKYNCVKIMFVGENIVPDFNLCDYAMGFDFLNFGDRYMRLPLFLICDNFKELSVTKDFSPKRMLNRKFCSIVVSNAQVSNPIRERFFRLLSEYKQVDSGGRLWNNVGGPVADKQKFISGYKFNIAFENSAVLGYTTEKIMDAMTANTLPIYWGNPWVGRDFNKRSFVNVNSFASLEKAVEYIVDLDTNDERYLEMMQEPWVNDVSIFDWEDKLCAFLAHIVEKPFAEAQYLVDDGMQKLYKQNMKTLAFVNEKLKVPRLISAYKKLSNGKYK

SEQ ID NO: 6 - DNA, 942 bp, synthetic construct
  source 1..942: mol_type = other DNA
atgaaaaccatcaagctgaaatataccgatttctgggatcatttcgacccgaaaaacaactttattaccaatgtgctgagcaaaaagtaccacatcgaatttagcgatacaccggattatctgatctttagcatctttggctatgagcacctgaaattccagaactgcattaaaatcctgtatagcggtgaaaacatcacaccggactttaatctgtgtgattatgccattggctttgaccatattacctttggtgatcgttatatgcgtctgccgctgtatgttagctatgatattgaacgtctggcaaaaccgaaagcaattgatccggaaaaagtgctgaatcgcaaattttgcagctttgttgttagcaatggtgttagtccggaacgtaatcgtttctttaaactgctgagcgaatataaacgtgttgatagcggtggtcgctttatgaataacattggtggtccggttccggataaagaagcattcatcaaagactataaattcaacattgccttcgagaacagcatctatgatggttataccaccgaaaaactgctggaaccgatgctgatggatagcctgccgatttattggggtaataaactggttggtctggattttaacccgcagagctttattaacgcaaccgattttccgagcctggaagcagttgttgaatatgttgttaaactggataccaacgatgaggactatctgagcaaactgagcattccgtggctgaacaaaaataactatctgaattgggagagccagttctatgatttcttcgataacatctttaacaaaccgaaaagcgaagcacgttatctgaccgattatggtcgtgttcgtatttatcgtcagcgtctgaacctgtttagcaccattgatgtgattaaacgcaaactggatgatctgcgtaatcgctttcgctaa

SEQ ID NO: 7 - DNA, 972 bp, synthetic construct
  source 1..972: mol_type = other DNA
atgaacaacaagatcaagatcaacttcgtggatttctggggtgatttcaacaaatgcgataactttattacccgtgcgctgagcaaaaagtatgaagttgcaattagcgataacccggattacctgtttttcggcacctttggttatcgtcatctgaattataactgcgtgaaaatcatgttcatcggcgaaaatattgcaccggattttaacctgtgtgattatgcactgggctttgatttcattcagtttggtgatcgttatatgcgtctgccgctgtattgtacctatgaaagctttgcaagcctggcacagacccgtctgccgagtgatgaagcactgattgatcgtaaattttgcagcatggtggtgagcaataatcgtcatgcaagtccgcatcgtgaacgtttctttaaactgctgagcgaatataaacaggttgatagcggtggtcgtctgtggaataatgttggtggtccggttgcaaataaactggaatttgttagccagtacaaattcaacattgcctttgaaaatagcgcagtgcatggttataccaccgagaagattatggaaccgatggcagttaataccctgccgttatattggggtaatccgctggtttgcaaagactttaatgcagccagctttgtgaacgtgaatgattttgcaagtatggaagatgccgttcagcatattgttgatctggataacgatgacagcaaatacctgcagatgattaaagcaccgaaagtgctggatgaaaacatcattaattgggaagaacgcctgctggattttctgagcaatattatcgaaaaaccgatcgaggaagcacgttatctggcaccgtttggtgttcagaaactgtatcgtgaagaactgctggcatatgatattctgagccgtcgtctgaaagtgaaaaagattgttgcagcctatgataaactgcgcaaaaagattcgtggctaa

SEQ ID NO: 8 - DNA, 963 bp, synthetic construct
  source 1..963: mol_type = other DNA
atgcgtgatgttaccgttgcctttgttgatttttggcctgcctttgatgacaaaaacaactttattctggatgtgctgcgccagaaatataacgttaaaaccgttgcagcaagcgattgtccggatattctgttttatagctatttcggcattgaaaacctgcgtcataccgaaccggtgcgtgtgtattttacaggtgaaaatgatgtgccggatttcaacctgtgtgattatgcaattagctttcaccatctgagctttgcaaatcgtcatctgcgtctgccgctgtatgtgatttatagctgttttaatgatctgcgcaatggtcgtattctgcagtgtgaagatgcagaaaatcgtgatttttgcagcatggttgtgagcaatgtgaaaaatagcgatatggcacgtctggatttctttaatctgctgagcggttataaacaggttgcaagcggtggtcgttttgcaaataatgttggtggttgtgttaccgacaaactgagctttattagcggctacaaattcaacattgcctttgaaaattcacgcgtggaaggttatgtgaccgagaaaattatcgaagcatttagcagcggcaccctgccgatttattggggtgatccgctggttgcaaatgaactgaatgataaagccttcatcgacgtgagcaaatttgatagcctggatgcagcagttaaatttgttgcagcactggataataaccgcgaacagtatctggaaattctgaatcagcctccgctgctgaataatccgtttctgaattgggaagaaatgctgctggattttctgaccccgattgttaacgaactgaaaaagtatcgtgttgataccggtgcaattggtacagcatatcgtcataaaatgagccgtgagcagatttataacattgtgaccctgaaaaagccgttcgaactgttcaaacacctgtttcacagctaa

SEQ ID NO: 9 - DNA, 1044 bp, synthetic construct
  source 1..1044: mol_type = other DNA
atgccgaaaaagcacatcaaaatcctgtttgtggatttttgggataactttagcccgaccgataacctgtttgttaatctgctgagtgatgcctatgatatcgaagttagcgaaacaccggattacctgttttatagcgtttttggtcagaaacacctgaaatatcgtgattgcgtgcgtatcttttataccggtgaaaatcagagtccggattttaacgtttgtgattatgccgtgggctttgatcacattacctttatggatcgctatcatcgttttccgctgtatgttcagtttgccggtgattatgatgacatgatgagcaaacatgcacgtaccgatggtgaactgctggcaggtcgtgatggtttttgtagctttgtttatagcaacaataacgccagtccggaacgtaatcgtttttatgatctgctgtgtacctataaaccggttgcaagcggtggtcgttttcgtaataatgttggtggtccggttgcagataagaaagaatttcagagccattatcgttttagcgtggcctttgaaaataccagcgttccgggttatgcaaccgaaaaactggttcaggcatttgcagcaggcaccgttccggtttattggggtgatccgctgattgatcgtacctttaatagcaaagcctttatcaactgccacgattatgatagctttgaagatgttctggcagcagttcgtcgtattgatcaggatgaaagcctgtatctggcaatgctgcgtgaaccggcactgattgatatgaatgatacccgtgaacgtcgtctggaagaactgcgtagctttctgtgtcatattctggatcaggatccggttagcgcacgtcgttatagccgtgaatattgggcactgcgttattatgataatctggaagttcgtgaaaccgtgtatcgtcgtagcctgcgtggtattgcagaaagcttctataaaaagtatttttggcgttggcgtcgtagcagccgtctgatgtggaaaattgatcgttttctgaaacgtctgatcggtcgctaa

SEQ ID NO: 10 - DNA, 966 bp, synthetic construct
  source 1..966: mol_type = other DNA
atgaaaagcctgaagatcaagttcgtggatttttggcctggttttaatccgaacgataactttattaccaatgcgctgaaaggctatgagattgtgattaccgatacaccggattacctgtttttcagcatttttggttatagccacctgaagtacaactgcgtgaaaattatgtttgtgggcgaaaatatcgtgccggattttaatctgtgtgattatgccatgggcttcgactttctgaattttggtgatcgttatatgcgtctgccgctgtttctgatttgcgataatttcaaagaactgagcgtgaccaaagattttagcccgaaacgtatgctgaatcgcaaattttgtagcattgttgttagcaatgcccaggttagcaatccgattcgtgaacgtttctttcgtctgctgagcgaatataaacaggttgatagcggtggtcgtctgtggaataatgttggtggtccggttgcagataaacagaaatttatctccggctataaattcaacattgcctttgaaaatagcgcagtgctgggttataccaccgaaaagattatggatgcaatgaccgcaaataccctgccgatttattggggtaatccgtgggttggtcgtgatttcaataaacgtagctttgtgaacgtgaatagctttgcaagcctggaaaaagccgttgaatatattgttgatctggataccaacgatgagcgctatctggaaatgatgcaagaaccgtgggttaatgatgtgtccatttttgactgggaagataaactgtgtgcatttctggcacatattgtcgaaaaaccgtttgcagaagcacagtatctggttgatgatggtatgcagaaactgtacaagcagaatatgaaaaccctggcctttgtgaatgagaaactgaaagttccgcgtctgattagcgcatataagaaactgagcaacggcaagtacaaataa

SEQ ID NO: 11 - DNA, 6706 bp, synthetic construct
  source 1..6706: mol_type = other DNA
atgtcaaaagtcgctctcatcaccggtgtaaccggacaagacggttcttacctggcagagtttctgctggaaaaaggttacgaggtgcatggtattaagcgtcgcgcatcgtcattcaacaccgagcgcgtggatcacatttatcaggatccgcacacctgcaacccgaaattccatctgcattatggcgacctgagtgatacctctaacctgacgcgcattttgcgtgaagtacagccggatgaagtgtacaacctgggcgcaatgagccacgttgcggtctcttttgagtcaccagaatataccgctgacgtcgacgcgatgggtacgctgcgcctgctggaggcgatccgcttcctcggtctggaaaagaaaactcgtttctatcaggcttccacctctgaactgtatggtctggtgcaggaaattccgcagaaagagaccacgccgttctacccgcgatctccgtatgcggtcgccaaactgtacgcctactggatcaccgttaactaccgtgaatcctacggcatgtacgcctgtaacggaattctcttcaaccatgaatccccgcgccgcggcgaaaccttcgttacccgcaaaatcacccgcgcaatcgccaacatcgcccaggggctggagtcgtgcctgtacctcggcaatatggattccctgcgtgactggggccacgccaaagactacgtaaaaatgcagtggatgatgctgcagcaggaacagccggaagatttcgttatcgcgaccggcgttcagtactccgtgcgtcagttcgtggaaatggcggcagcacagctgggcatcaaactgcgctttgaaggcacgggcgttgaagagaagggcattgtggtttccgtcaccgggcatgacgcgccgggcgttaaaccgggtgatgtgattatcgctgttgacccgcgttacttccgtccggctgaagttgaaacgctgctcggcgacccgaccaaagcgcacgaaaaactgggctggaaaccggaaatcaccctcagagagatggtgtctgaaatggtggctaatgacctcgaagcggcgaaaaaacactctctgctgaaatctcacggctacgacgtggcgatcgcgctggagtcataagcatgagtaaacaacgagtttttattgctggtcatcgcgggatggtcggttccgccatcaggcggcagctcgaacagcgcggtgatgtggaactggtattacgcacccgcgacgagctgaacctgctggacagccgcgccgtgcatgatttctttgccagcgaacgtattgaccaggtctatctggcggcggcgaaagtgggcggcattgttgccaacaacacctatccggcggatttcatctaccagaacatgatgattgagagcaacatcattcacgccgcgcatcagaacgacgtgaacaaactgctgtttctcggatcgtcctgcatctacccgaaactggcaaaacagccgatggcagaaagcgagttgttgcagggcacgctggagccgactaacgagccttatgctattgccaaaatcgccgggatcaaactgtgcgaatcatacaaccgccagtacggacgcgattaccgctcagtcatgccgaccaacctgtacgggccacacgacaacttccacccgagtaattcgcatgtgatcccagcattgctgcgtcgcttccacgaggcgacggcacagaatgcgccggacgtggtggtatggggcagcggtacaccgatgcgcgaatttctgcacgtcgatgatatggcggcggcgagcattcatgtcatggagctggcgcatgaagtctggctggagaacacccagccgatgttgtcgcacattaacgtcggcacgggcgttgactgcactatccgcgagctggcgcaaaccatcgccaaagtggtgggttacaaaggccgggtggtttttgatgccagcaaaccggatggcacgccgcgcaaactgctggatgtgacgcgcctgcatcagcttggctggtatcacgaaatctcactggaagcggggcttgccagcacttaccagtggttccttgagaatcaagaccgctttcgggggtaatgatgtttttacgtcaggaagactttgccacggtagtgcgctccactccgcttgtctctctcgactttattgtcgagaacagtcgcggcgagtttctgcttggcaaaagaaccaaccgcccggcgcagggttactggtttgtgccgggagggcgcgtgcagaaagacgaaacgctggaagccgcatttgagcggctgacgatggcggaactggggctgcgtttgccgataacagcaggccagttttacggtgtctggcagcacttttatgacgataacttctctggcacggatttcaccactcactatgtggtgctcggttttcgcttcagagtatcggaagaagagctgttactgccggatgagcagcatgacgattaccgctggctgacgtcggacgcgctgctcgccagtgataatgttcatgctaacagccgcgcctattttctcgctgagaagcgtaccggagtacccggattatgaaaatactggtctacggcattaactactcgccggagttaaccggcatcggcaaatacaccggcgagatggtggaatggctggcggcacaaggtcatgaggtgcgggtcattaccgcaccgccttactacccgcaatggcaggtgggcgagaactattccgcctggcgctacaaacgagaagagggggccgccacggtgtggcgctgcccgctgtatgtgccaaaacagccgagcaccctgaaacgcctgttgcatctgggcagttttgccgtcagcagtttctttccgctgatggcgcaacgtcgctggaagccggatcgcattattggcgtggtgccaacgctgttttgcgcgccgggaatgcgcctgctggcgaaactctctggtgcgcgtaccgtgctgcatattcaggattacgaagtggacgccatgctggggctgggccttgccggaaaaggcaaaggcggcaaagtggcacagctggcaacggcgttcgaacgtagcggactgcataacgtcgataacgtctccacgatttcgcgttcgatgatgaataaagccatcgaaaaaggcgtggcggcggaaaacgtcatcttcttccccaactggtcggaaattgcccgttttcagcatgttgcagatgccgatgttgatgcccttcgtaaccagcttgacctgccggataacaaaaaaatcattctttactccggcaatattggtgaaaagcaggggctggaaaacgttattgaagctgccgatcgtctgcgcgatgaaccgctgatttttgccattgtcgggcagggcggcggcaaagcgcggctggaaaaaatggcgcagcagcgtggactgcgcaacatgcaatttttcccgctgcaatcgtatgacgctttacccgcactgctgaagatgggcgattgccatctggtggtgcaaaaacgcggcgcggcagatgccgtattgccgtcgaaactgaccaatattctggcagtaggcggtaacgcggtgattactgctgaagcctacacagaactggggcagctttgcgaaacctttccgggcattgcggtttgcgttgaaccggaatcggtcgaggcgctggtggcggggatccgtcaggcgctcctgctgcccaaacacaacacggtggcacgtgaatatgccgaacgcacgctcgataaagagaacgtgttacgtcaatttataaatgatattcggggataattatggcgcagtcgaaactctatccagttgtgatggcaggtggctccggtagccgcttatggccgctttcccgcgtactttatcccaagcagtttttatgcctgaaaggcgatctcaccatgctgcaaaccaccatctgccgcctgaacggcgtggagtgcgaaagcccggtggtgatttgcaatgagcagcaccgctttattgtcgcggaacagctgcgtcaactgaacaaacttaccgagaacattattctcgaaccggcagggcgaaacacggcacctgccattgcgctggcggcgctggcggcaaaacgtcatagcccggagagcgacccgttaatgctggtattggcggcggatcatgtgattgccgatgaagacgcgttccgtgccgccgtgcgtaatgccatgccatatgccgaagcgggcaagctggtgaccttcggcattgtgccggatctaccagaaaccggttatggctatattcgtcgcggtgaagtgtctgcgggtgagcaggatatggtggcctttgaagtggcgcagtttgtcgaaaaaccgaatctggaaaccgctcaggcctatgtggcaagcggcgaatattactggaacagcggtatgttcctgttccgcgccggacgctatctcgaagaactgaaaaaatatcgcccggatatcctcgatgcctgtgaaaaagcgatgagcgccgtcgatccggatctcaattttattcgcgtggatgaagaagcgtttctcgcctgcccggaagagtcggtggattacgcggtcatggaacgtacggcagatgctgttgtggtgccgatggatgcgggctggagcgatgttggctcctggtcttcattatgggagatcagcgcccacaccgccgagggcaacgtttgccacggcgatgtgattaatcacaaaactgaaaacagctatgtgtatgctgaatctggcctggtcaccaccgtcggggtgaaagatctggtagtggtgcagaccaaagatgcggtgctgattgccgaccgtaacgcggtacaggatgtgaaaaaagtggtcgagcagatcaaagccgatggtcgccatgagcatcgggtgcatcgcgaagtgtatcgtccgtggggcaaatatgactctatcgacgcgggcgaccgctaccaggtgaaacgcatcaccgtgaaaccgggcgagggcttgtcggtacagatgcaccatcaccgcgcggaacactgggtggttgtcgcgggaacggcaaaagtcaccattgatggtgatatcaaactgcttggtgaaaacgagtccatttatattccgctgggggcgacgcattgcctggaaaacccggggaaaattccgctcgatttaattgaagtgcgctccggctcttatctcgaagaggatgatgtggtgcgtttcgcggatcgctacggacgggtgtaaacgtcgcatcaggcaatgaatgcgaaaccgcggtgtaaataacgacaaaaataaaattggccgcttcggtcagggccaactattgcctgaaaaagggtaacgatatgaaaaaattaacctgctttaaagcctatgatattcgcgggaaattaggcgaagaactgaatgaagatatcgcctggcgcattggtcgcgcctatggcgaatttctcaaaccgaaaaccattgtgttaggcggtgatgtccgcctcaccagcgaaaccttaaaactggcgctggcgaaaggtttacaggatgcgggcgttgacgtgctggatattggtatgtccggcaccgaagagatctatttcgccacgttccatctcggcgtggatggcggcattgaagttaccgccagccataatccgatggattataacggcatgaagctggttcgcgagggggctcgcccgatcagcggagataccggactgcgcgacgtccagcgtctggctgaagccaacgactttcctcccgtcgatgaaaccaaacgcggtcgctatcagcaaatcaacctgcgtgacgcttacgttgatcacctgttcggttatatcaatgtcaaaaacctcacgccgctcaagctggtgatcaactccgggaacggcgcagcgggtccggtggtggacgccattgaagcccgctttaaagccctcggcgcgcccgtggaattaatcaaagtgcacaacacgccggacggcaatttccccaacggtattcctaacccactactgccggaatgccgcgacgacacccgcaatgcggtcatcaaacacggcgcggatatgggcattgcttttgatggcgattttgaccgctgtttcctgtttgacgaaaaagggcagtttattgagggctactacattgtcggcctgttggcagaagcattcctcgaaaaaaatcccggcgcgaagatcatccacgatccacgtctctcctggaacaccgttgatgtggtgactgccgcaggtggcacgccggtaatgtcgaaaaccggacacgcctttattaaagaacgtatgcgcaaggaagacgccatctatggtggcgaaatgagcgcccaccattacttccgtgatttcgcttactgcgacagcggcatgatcccgtggctgctggtcgccgaactggtgtgcctgaaagataaaacgctgggcgaactggtacgcgaccggatggcggcgtttccggcaagcggtgagatcaacagcaaactggcgcaacccgttgaggcgattaaccgcgtggaacagcattttagccgtgaggcgctggcggtggatcgcaccgatggcatcagcatgacctttgccgactggcgctttaacctgcgcacctccaataccgaaccggtggtgcgcctgaatgtggaatcgcgcggtgatgtgccgctgatggaagcgcgaacgcgaactctgctgacgttgctgaacgagtaa

SEQ ID NO: 12 - protein, 417 aa, Escherichia coli [sequence omitted]

SEQ ID NO: 13 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagaaaaacagct

SEQ ID NO: 14 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactgagaaacagct

SEQ ID NO: 15 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaacaaaaaccggagatacc

SEQ ID NO: 16 - DNA, 141 bp, synthetic construct
  source 1..141: mol_type = other DNA
tgtgagttagctcactcattaggcaccccaggctttacactttatgcttccggctcgtatgttgtgtggaatgcctacaagcatcgtggaggtccgtgactttcacgcatacaacaaacattaaccaaggaggaaacagct

SEQ ID NO: 17 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaaggaaaaacagct

SEQ ID NO: 18 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactaggaaacagct

SEQ ID NO: 19 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaccgagaaacagct

SEQ ID NO: 20 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactaggaaacagct

SEQ ID NO: 21 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagcaaaacagct

SEQ ID NO: 22 - DNA, 203 bp, synthetic construct
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaggaggaaacagct

SEQ ID NO: 23 - DNA, 189 bp, synthetic construct
  source 1..189: mol_type = other DNA
gaaaacattcataaattaaatgtgaattgccgcacacattattaaataagatttacaaaatgttcaaaatgacgcatgaaatcacgtttcactttcgaattatgagcgaatatgcgcgatgcctacaagcatcgtggaggtccgtgactttcacgcatacaacaaacattaaccaaggaggaaacagct

SEQ ID NO: 24 - DNA, 239 bp, synthetic construct
  source 1..239: mol_type = other DNA
ccatttagccatagtaaaaacatgaattgtttgatttcgcgcatattcgctcataattcgaaagtgaaacgtgatttcatgcgtcattttgaacattttgtaaatcttatttaataatgtgtgcggcaattcacatttaatttatgaatgttttcttaacatcgcggcatgcctacaagcatcgtggaggtccgtgactttcacgcatacaacaaacattaaccaaggaggaaacagct

SEQ ID NO: 25 - DNA, 300 bp, Escherichia coli
  source 1..300: mol_type = genomic DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaggaggaaacagct

SEQ ID NO: 26 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactgagaaacagct

SEQ ID NO: 27 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaccgagaaacagct

SEQ ID NO: 28 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagaaaaacagct

SEQ ID NO: 29 - DNA, 350 bp, synthetic construct
  source 1..350: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgttttaacgttgtaacccgtatgtaacagtgaataatcacttttgccgaggtaacagcgtcataacaacaattaaagccgttttctggagcgttaccgggcatggaagaacgaattttaaaaagtgagcttcggcgttcagtaacacttcattaactctactgccccgccgagcatttatctcaagcactaccctgcataagcaaggaggaaacagct

SEQ ID NO: 30 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaaggaaaaacagct

SEQ ID NO: 31 - DNA, 300 bp, synthetic construct
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagcaaaacagct

SEQ ID NO: 32 - DNA, 310 bp, synthetic construct
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagctaaacagct

SEQ ID NO: 33 - DNA, 189 bp, synthetic construct; identical to SEQ ID NO: 23
  source 1..189: mol_type = other DNA

SEQ ID NO: 34 - DNA, 107 bp, Escherichia coli
  source 1..107: mol_type = genomic DNA
tgtgagttagctcactcattaggcaccccaggctttacactttatgcttccggctcgtatgttgtgtggaattgtgagcggataacaatttcacacaggaaacagct

SEQ ID NO: 35 - DNA, 300 bp, synthetic construct
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagaacaaaacagct

SEQ ID NO: 36 - DNA, 300 bp, synthetic construct
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaattcgaaacagct

(19) United States
(12) Patent Application Publication
(10) Pub. No.: US 2026/0226483 A1
(43) Pub. Date: Aug. 6, 2026

(54) NEW FUCOSYLTRANSFERASES FOR PRODUCTION OF 3FL

(71) Applicant: DSM IP Assets B.V., Maastricht (NL)

(72) Inventor: Manos PAPADAKIS, Hørsholm (DK)

(21) Appl. No.: 19/142,047

(22) PCT Filed: Dec. 21, 2023
(86) PCT No.: PCT/EP2023/087291

§ 371 Date: Jun. 20, 2025

(30) Foreign Application Priority Data
Dec. 22, 2022 (DK) PA202201204

(51) Int. Cl. C12N 15/70; C12N 9/10; C12P 19/18; C12R 1/19

(52) U.S. Cl. CPC C12N 15/70; C12N 9/1051; C12P 19/18; C12R 2001/19; C12Y 204/01065

(57) ABSTRACT

The present disclosure relates to the biosynthetic production of the fucosylated Human Milk Oligosaccharides (HMO), 3-fucosyllactose, as well as to genetic engineering cells and α-1,3-fucosyltransferases suitable for use in said biosynthetic production, as well as to methods for producing 3FL.

### CROSS-REFERENCE TO RELATED APPLICATIONS

This application is a national stage entry pursuant to 35 U.S.C. § 371 of International Patent Application No. PCT/EP2023/087291, filed on Dec. 21, 2023, which claims priority to Denmark Patent Application No. PA202201204, filed on Dec. 22, 2022, the entire contents of all of which are hereby incorporated by reference in their entirety.

### SEQUENCE LISTING

The computer-readable Sequence Listing submitted on Apr. 23, 2026, and identified as follows: 51,597 bytes ST.26 XML document file named “Sequence-Listing.xml,” created Mar. 18, 2026, is incorporated herein by reference in its entirety.

### FIELD

The present disclosure relates to the biosynthetic production of the fucosylated Human Milk Oligosaccharide (HMO), 3-fucosyllactose (3FL), and to genetically engineered cells suitable for use in said biosynthetic production of 3FL.

### BACKGROUND

The construction of bacterial cell factories to produce Human Milk Oligosaccharides (HMOs), especially fucosylated Human Milk Oligosaccharides (HMOs) started in the early 2000 and has continuously developed over the last two decades as reviewed by Petschacher and Nidetzky 2016 Journal of Biotechnology 235: 61-83 and Bych et al 2019, Current Opinion in Biotechnology 56:130-137.

The human milk oligosaccharide 3FL (3-fucosyllactose) is among the 10 most abundant HMOs in breast milk. Research has shown that the concentration of 3FL actually increases in breast milk during the length of the lactation phase. Thus, it is in particular of importance to identify new fucosyltransferases for HMO production that enable very high yields of 3FL, as well as a stable growth of the production strain, to obtain a robust fucosylated HMO production platform that is scalable for industrial production of 3FL.

None the less the identification of α-1,3-fucosyltransferases that can provide high 3FL yields when applied in vivo has proven to be challenge as highlighted by Chen et al. 2022 J. Agric. Food Chem., 70, 6, 1934-1942), which describes the search for novel α-1,3-fucosyltransferases useful in efficient production of 3FL including improving 3FL titers. Chen et al. describes the identification of the fucosyltransferase FutM2 from Bacteroides gallinaceum, herein referred to as Bgall1, which when expressed from a plasmid in an engineered Escherichia coli strain, overexpressing the de novo GDP-fucose pathway is reported to produce 20 g/L of 3FL from glycerol as carbon source.

WO 2016/040531 also discloses α-1,3-fucosyltransferases for the production of fucosylated HMOs. A number of these however appear to have α-1,2-fucosyltransferase activity as well since they appear to also produce difucosyllactose (DFL or LDFT) as well.

In industrial scale even an increase of production yields of a few % is of high value, there is therefore a continuous interest in identifying enzymes which are even better than FutM2/Bgall1 from Bacteroides gallinaceum.

### SUMMARY

Provided herein are enzymes, uses, genetically engineered cells and methods for the production of 3-fucosyllactose.

A first aspect, relates to a genetically engineered cell capable of producing 3FL, comprising a recombinant nucleic acid sequence encoding a fucosyltransferase with α-1,3-fucosyltransferase activity wherein the glycosyltransferase is selected from the group consisting of,
a. Osc1, comprising or consisting of the amino acid sequence of SEQ ID NO: 1, or a functional homologue thereof with an amino acid sequence that is at least 80% identical to SEQ ID NO: 1,
b. Bbac1 comprising or consisting of the amino acid sequence of SEQ ID NO: 2, or a functional homologue thereof with an amino acid sequence that is at least 80% identical to SEQ ID NO: 2,
c. Murba1 comprising or consisting of the amino acid sequence of SEQ ID NO: 3, or a functional homologue thereof with an amino acid sequence that is at least 80% identical to SEQ ID NO: 3 and
d. Bacbac1 comprising or consisting of the amino acid sequence of SEQ ID NO: 4, or a functional homologue thereof with an amino acid sequence that is at least 80% identical to SEQ ID NO: 4.

Production of 3FL requires that the α-1,3-fucosyltransferase used is capable of fucosylating lactose at the Glu moiety in an α-1,3 position. Accordingly, the α-1,3-fucosyltransferase results in formation of 3FL when lactose is the substrate.

A second aspect, relates to a method for producing 3FL, said method comprising providing and cultivating a genetically engineered cell comprising a recombinant nucleic acid sequence encoding a fucosyltransferase with α-1,3-fucosyltransferase activity, wherein the glycosyltransferase is selected from the group consisting of, a) Osc1, comprising or consisting of the amino acid sequence of SEQ ID NO: 1, or a functional homologue thereof with an amino acid sequence that is at least 80% identical to SEQ ID NO: 1, b) Bbac1 comprising or consisting of the amino acid sequence of SEQ ID NO: 2, or a functional homologue thereof with an amino acid sequence that is at least 80% identical to SEQ ID NO: 2, c) Murba1 comprising or consisting of the amino acid sequence of SEQ ID NO: 3, or a functional homologue thereof with an amino acid sequence that is at least 80% identical to SEQ ID NO: 3 and d) Bacbac1 comprising or consisting of the amino acid sequence of SEQ ID NO: 4, or a functional homologue thereof with an amino acid sequence that is at least 80% identical to SEQ ID NO: 4.

A third aspect, relates to the use of a fucosyltransferase with α-1,3-fucosyltransferase activity in the production of 3FL, wherein the enzyme is selected from the group consisting of Osc1, Bbac1, Murba1, and Bacbac1 comprising or consisting of the amino acid sequence of SEQ ID NO: 1, 2, 3 or 4, or a functional homologue thereof with an amino acid sequence that is at least 80% identical to SEQ ID NO: 1, 2, 3, or 4.

### DETAILED DESCRIPTION

The present disclosure approaches the opportunities to improve biotechnological approaches for in vivo HMO production (i.e., biosynthetic HMO production), in particular of fucosylated HMOs that contain at least one fucosyl monosaccharide, such as the fucosylated HMOs 3FL and DFL.

The present disclosure offers specific strain engineering solutions to produce specific fucosylated HMOs, in particular 3FL, by exploiting the substrate specificity towards the glucose (Glc) moiety on lactose and activity of the α-1,3-fucosyltransferases of the present disclosure, which improves the production of fucosylated HMOs through increased production yields. Preferably, the α-1,3-fucosyltransferases of the present disclosure possess very limited, such as no α-1,2-fucosyltransferase activity. The lack of α-1,2-fucosyltransferase activity will lead to a pure 3FL product with essentially no or low amounts of DFL by-product as observed for other α-1,3-fucosyltransferases.

In essence, the present disclosure describes several newly identified enzymes with α-1,3-fucosyltransferase activity that produce higher amounts of 3FL, compared to the enzyme Bgall1 (see example 1). Bgall1 has been reported to produce about 20 g/L of 3FL in a strain with the conventional improvements used in strains to improve fucosyllactose formation. Such improvements namely comprises overexpression of the de novo GDP-fucose pathway genes responsible for the formation of GDP-fucose, which is needed to provide sufficient donor substrate for the fucosyllactose (i.e., 3FL) formation, along with deletion of wcaJ, to prevent GDP-fucose degradation, and deletion of IacZ to prevent degradation of lactose, which acts as the acceptor in the production of fucosyllactose (FIG. 1 of Chen et al. 2022 J. Agric. Food Chem., 70, 6, 1934-1942).

The enzymes presented herein thus serve as favourable alternatives to the enzyme Bgall1 for improved large-scale manufacturing of 3FL.

In the following sections, individual elements of the disclosure, and in particular of the genetically engineered cell are described. It is understood that these elements can be combined across the individual sections.

### Oligosaccharides

In the present context, the term “oligosaccharide” means a sugar polymer containing at least three monosaccharide units, i.e., a tri-, tetra-, penta-, hexa- or higher oligosaccharide. The oligosaccharide can have a linear or branched structure containing monosaccharide units that are linked to each other by interglycosidic linkages. Particularly, the oligosaccharide comprises a lactose residue at the reducing end and one or more naturally occurring monosaccharides of 5-9 carbon atoms selected from aldoses (e.g., glucose, galactose, ribose, arabinose, xylose, etc.), ketoses (e.g., fructose, sorbose, tagatose, etc.), deoxysugars (e.g. rhamnose, fucose, etc.), deoxy-aminosugars (e.g. N-acetyl-glucosamine, N-acetyl-mannosamine, N-acetyl-galactosamine, etc.), uronic acids and ketoaldonic acids (e.g. N-acetylneuraminic acid). Preferably, the oligosaccharide is an HMO.

### Human Milk Oligosaccharide (HMO)

Preferred oligosaccharides of the disclosure are human milk oligosaccharides (HMOs).

The term “human milk oligosaccharide” or “HMO” in the present context means a complex carbohydrate found in human breast milk. The HMOs have a core structure comprising a lactose unit at the reducing end that can be elongated by one or more beta-N-acetyl-lactosaminyl and/or one or more beta-lacto-N-biosyl unit, and this core structure can be substituted by an alpha-L-fucopyranosyl and/or an alpha-N-acetyl-neuraminyl (fucosyl) moiety. HMO structures are e.g., disclosed by Xi Chen in Chapter 4 of Advances in Carbohydrate Chemistry and Biochemistry 2015 vol 72.

The present disclosure focuses on fucosylated HMO's. Examples of fucosylated HMOs include, 2′-fucosyllactose (2′FL), lacto-N-fucopentaose I (LNFP-I), lacto-N-difucohexaose I (LNDFH-I), 3-fucosyllactose (3FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-fucopentaose VI (LNFP-VI), lacto-N-difucohexaose II (LNDFH-II), fucosyl-lacto-N-hexaose I (FLNH-I), fucosyl-para-lacto-N-hexaose I (FpLNH-1), fucosyl-para-lacto-N-neohexaose II (F-pLNnH II), fucosyl-lacto-N-neohexaose (FLNnH), 3-fucosyl-3′-fucosyllactose (FSL), fucosyl-LST-a (FLST-a), fucosyl-LST b (FLST b), fucosyl-LST-c (FLST-c), fucosyl-LST d (FLST-d) and fucosyl-lacto-N-hexaose (SLNH).

In embodiments of the present disclosure, the α-1,3-fucosyltransferases disclosed herein predominantly fucosylates the glucose (Glc) moiety of lactose. In that regard the α-1,3-fucosyltransferases disclosed herein should be capable of producing 3FL in the presence of a fucosyl donor and an acceptor being lactose. In addition, the α-1,3-fucosyltransferases disclosed herein may also be capable of producing additional fucosylated HMOs such as but not limited to fucosylated HMOs with an LNT or LNnT backbone. Fucosylated HMOs with an LNT or LNnT backbone may in the regard be lacto-N-fucopentaose I (LNFP-1), lacto-N-fucopentaose II (LNFP-II), lacto-N-fucopentaose III (LNFP-III), lacto-N-fucopentaose V (LNFP-V), lacto-N-fucopentaose VI (LNFP-VI), Lacto-N-difucohexaose I (LNDFH-1), Lacto-N-difucohexaose II (LNDFH-II) and Lacto-N-difucohexaose III (LNDFH-III).

In preferred embodiments the α-1,3-fucosyltransferases as disclosed herein produces HMOs with an α-1,3-linked fucosyl moiety on the terminal glucose moiety of the HMO such as in 3FL, LNFP-V or LNFP-VI. In addition, some of the α-1,3-fucosyltransferases as disclosed herein may also be able to fucosylate the GlcNAc moiety in oligosaccharides with an LNT backbone to produce LNFP-III or LNDFH-III. Furthermore, some of the enzymes may also possess α-1,4-fucosyltransferase activity and optionally produce oligosaccharides with an α-1,4-linked fucosyl moiety in addition to the α-1,3-linked fucosyl moiety such as LNDFH-II.

In human milk, about 60% of the content of HMOs are fucosylated HMOs wherein 3FL is the third most abundant fucosylated HMO in human milk with a 3FL content of approximately 5% of the total HMO content in human milk (Bych et al. 2019, Current Opinion in Biotechnology 56:130-137), thus production of mixtures comprising a high content of fucosylated HMOs is highly desirable for the production of more natural mixtures of HMOs.

### An Acceptor Oligosaccharide

A genetically engineered cell according to the present disclosure comprises a recombinant nucleic acid sequence encoding an α-1,3-fucosyltransferase capable of transferring fucose from an activated sugar to the glucose (Glc) moiety of an acceptor oligosaccharide, such as lactose, in an α-1,3 linkage. The α-1,3-fucosyltransferase may also be capable of transferring a fucosyl moiety to e.g., the Glc, N-acetylglucoseamine (GlcNAc) and/or galactose (Gal) moiety of more complex acceptor oligosaccharides, such as oligosaccharides with a lacto-N-triose II backbone, including LNT or LNnT and fucosylated or sialylated versions thereof.

In the context of the present disclosure, an acceptor oligosaccharide is an oligosaccharide (including disaccharides) that can act as a substrate for a glycosyltransferase capable of transferring a glycosyl moiety from a glycosyl donor to the acceptor oligosaccharide. The glycosyl donor is preferably a nucleotide-activated sugar as described in the section on “Glycosyl-donor—nucleotide-activated sugar pathways”. Preferably, the acceptor oligosaccharide is a precursor for making an HMO and can also be termed the precursor molecule.

The acceptor oligosaccharide can be either an intermediate product of the present fermentation process, an end-product of a separate fermentation process employing a separate genetically engineered cell, or an enzymatically or chemically produced molecule.

In the present context, said acceptor oligosaccharide is preferably lactose for the production of 3FL. The production of more complex HMOs than 3FL, such as LNFP-II, LNFP-III, LNFP-V, LNFP-VI, LNDFH-II and/or LNDFH-III may also include more complex acceptor molecules such as lacto-N-tetraose (LNT) or (lacto-N-neotetraose) LNnT, which are produced from the precursor molecules lactose and/or lacto-N-triose II (LNT-II). In both cases, the precursor molecule can be fed to the genetically modified cell which is capable of producing the fucosylated HMO from the precursor or the cell can be modified to produce the more complex acceptor molecule from lactose.

The acceptor oligosaccharide can be either an intermediate product of the present fermentation process, an end-product of a separate fermentation process employing a separate genetically modified cell, or an enzymatically or chemically produced molecule.

### Glycosyltransferases

The genetically engineered cell according to the present disclosure comprises at least one recombinant nucleic acid sequence encoding at least one glycosyltransferase, i.e., a fucosyltransferase, capable of transferring a fucosyl residue from a fucosyl donor to an acceptor oligosaccharide to synthesize one or more fucosylated human milk oligosaccharide products.

For production of 3FL the genetically engineered cell as described herein, preferably only contain one glycosyltransferase, selected from the α-1,3-fucosyltransferases described herein.

The term “α-1,3-fucosyltransferase” refers to a glycosyltransferase that catalyzes the transfer of fucosyl moiety from a donor substrate, such as GDP-fucose, to an acceptor molecule, such as lactose, in an α-1,3-linkage.

Preferably, an α-1,3-fucosyltransferase used in the present disclosure does not originate in the species of the genetically engineered cell, i.e., the gene encoding the α-1,3-fucosyltransferase is of heterologous origin and is selected from α-1,3-fucosyltransferases identified in table 1. In the context disclosed herein, the acceptor molecule for the α-1,3-fucosyltransferase is preferably lactose.

Heterologous α-1,3-fucosyltransferases which are capable of transferring a fucosyl moiety onto lactose are known in the art. Specifically Bgall1/FutM2, with an amino acid sequence as provided in SEQ ID NO: 5 and encoded by the nucleic acid sequence of SEQ ID NO: 10, is known to produce high titres of 3FL (Chen et al. 2022 J. Agric. Food Chem. 70, 6, 1934-1942).

An α-1,3-fucosyltransferase that produces high amounts of 3FL when the initial substrate is lactose is highly advantageous in large scale manufacturing of 3FL, as it lowers the overall production costs and increases productivity. In some embodiments the absence of by-products such as DFL is advantageous since this further reduces cost of purification.

As such, a genetically engineered cell which is capable of producing high titres of the intended product is highly advantageous as it simplifies the downstream processing and purification of the product substantially.

As can be seen in example 1 the α-1,3-fucosyltransferases disclosed herein all produce higher relative amounts of 3FL, than the known enzyme Bgall1/FutM2, which is known in the prior art to produce high titres of 3FL when introduced into a cell suitable for production of fucosylated HMOs.

In embodiments, a genetically engineered cell disclosed herein, expressing either Osc1, Bbac1, Murba1 or Bacbac1 produces at least 10% more, such as at least 15% more, such as at least 20% more 3FL than a genetically engineered cell expressing Bgall1.

The comparison between a α-1,3-fucosyltransferase described herein and Bgall, should be done in a strain with the same the same genetic background and with the enzymes being under control of identical promoters. Preferably the expression of Bgall1 is regulated via a PglpF promoter.

The α-1,3-fucosyltransferase can be selected from the α-1,3-fucosyltransferases with amino acid sequences with at least 80%, such as at least 85%, such as at least 90%, such as at least 95%, or such as at least 99% sequence identity to an amino acid sequence listed in table 1.

TABLE 1
List of α-1,3-fucosyltransferase
enzymes capable of producing 3FL.

| Enzyme | GenBank | SEQ | |
|---|---|---|---|
| Name | ID¹ | ID NO: | Origin |
| Osc1 | MBC8592593.1 | 1 | Oscillospiraceae bacterium N12 |
| Bbac1 | MBR5851405.1 | 2 | Bacteroidaceae bacterium |
| Murba1 | MBP3303338.1 | 3 | Muribaculaceae bacterium |
| Bacbac1 | MBQ0149235.1 | 4 | Bacteroidales bacterium |

¹The GenBank IDs reflect the full-length enzymes, in the present disclosure truncated, elongated or mutated versions may have been used, these are represented by the sequences indicated by the SEQ ID NOs.

Example 1 discloses the identification of the heterologous α-1,3-fucosyltransferases Osc1, Bbac1, Murba1 and Bacbac1 (SEQ ID NO: 1, 2, 3 and 4, respectively), which are all capable of producing the fucosylated HMO 3FL from lactose as the initial substrate (acceptor).

In one embodiment of the present disclosure, the functional enzyme (α-1,3-fucosyltransferase) capable of transferring a fucosyl moiety from a fucosyl donor to an acceptor oligosaccharide is selected from the group consisting of a) Osc1, comprising or consisting of the amino acid sequence of SEQ ID NO: 1, or a functional homologue thereof with an amino acid sequence that is at least 80% identical to SEQ ID NO: 1, b) Bbac1 comprising or consisting of the amino acid sequence of SEQ ID NO: 2, or a functional homologue thereof with an amino acid sequence that is at least 80% identical to SEQ ID NO: 2, c) Murba1 comprising or consisting of the amino acid sequence of SEQ ID NO: 3, or a functional homologue thereof with an amino acid sequence that is at least 80% identical to SEQ ID NO: 3 and d) Bacbac1 comprising or consisting of the amino acid sequence of SEQ ID NO: 4, or a functional homologue thereof with an amino acid sequence that is at least 80% identical to SEQ ID NO: 4.

Furthermore, experiments showed that the α-1,3-fucosyltransferases disclosed herein produced more than 20% more 3FL than Bgall1.

In one embodiment, the α-1,3-fucosyltransferase is Osc1 from Oscillospiraceae bacterium N12 comprising or consisting of the amino acid sequence of SEQ ID NO: 1, or an amino acid sequence with at least 80%, such as at least 85%, such as at least 90%, such as at least 95%, or such as at least 99% sequence identity to SEQ ID NO: 1. Preferably, expression of Osc1 results in a production of 3FL which is at least equal to, or at least 10%, such as at least 15%, such as at least 20%, such as at least 25%, or such as at least 30% higher than the amount of 3FL produced by a genetically modified cell expressing the α-1,3-fucosyltransferase Bgall1 (SEQ ID NO: 5), when the expression of Bgall1 is regulated via a PgIpF promoter. Expression of Osc1 in a strain capable of producing LNnT will also result in formation of significant amounts of LNDFH-III with insignificant amounts of LNFP-III and LNFP-VI. Furthermore, expression of Osc1 in a strain capable of producing LNT will also result in formation of significant amounts of LNFP-V.

In one embodiment, the α-1,3-fucosyltransferase is Bbac1 from Bacteroidaceae bacterium comprising or consisting of the amino acid sequence of SEQ ID NO: 2, or an amino acid sequence with at least 80%, such as at least 85%, such as at least 90%, such as at least 95%, or such as at least 99% sequence identity to SEQ ID NO: 2. Preferably, expression of Bbac1 results in a production of 3FL which is at least equal to, or at least 5%, such as at least 10%, such as at least 15% or such as at least 20% higher than the amount of 3FL produced by a genetically modified cell expressing the α-1,3-fucosyltransferase Bgall1 (SEQ ID NO: 5), when the expression of Bgall1 is regulated via a PgIpF promoter. Expression of Bbac1 in a strain capable of producing LNnT will also result in formation of significant amounts of LNDFH-III as well as both LNFP-III and LNFP-VI. Furthermore, expression of Bbac1 in a strain capable of producing LNT will also result in formation of both LNFP-V and LNDFH-II, indicating that this fucosyltransferase also have some α-1,4-fucosyltransferase activity.

In one embodiment the α-1,3-fucosyltransferase is Murba1 from Muribaculaceae bacterium comprising or consisting of the amino acid sequence of SEQ ID NO: 3, or an amino acid sequence with at least 80%, such as at least 85%, such as at least 90%, such as at least 95%, or such as at least 99% sequence identity to SEQ ID NO: 3. Preferably, expression of Murba1, results in a production of 3FL which is at least equal to, or at least 5%, such as at least 10%, such as at least 15% or such as at least 20% higher than the amount of 3FL produced by a genetically modified cell expressing the α-1,3-fucosyltransferase Bgall1 (SEQ ID NO: 5), when the expression of Bgall1 is regulated via a PgIpF promoter. Expression of Murba1 in a strain capable of producing LNnT will also result in formation of significant amounts of LNDFH-III and LNFP-III but very little LNFP-VI. Furthermore, expression of Murba1 in a strain capable of producing LNT will also result in formation of LNFP-V and some LNDFH-II, indicating that this fucosyltransferase also have some α-1,4-fucosyltransferase activity.

In one embodiment, the α-1,3-fucosyltransferase is Bacbac1 from Bacteroidales bacterium comprising or consisting of the amino acid sequence of SEQ ID NO: 4, or an amino acid sequence with at least 80%, such as at least 85%, such as at least 90%, such as at least 95%, or such as at least 99% sequence identity to SEQ ID NO: 4. Preferably, expression of Bacbac1 results in a production of 3FL which is at least equal to, or at least 5%, such as at least 10%, such as at least 15% or such as at least 20% higher than the amount of 3FL produced by a genetically modified cell expressing the α-1,3-fucosyltransferase Bgall1 (SEQ ID NO: 5), when the expression of Bgall1 is regulated via a PgIpF promoter. Expression of Bacbac1 in a strain capable of producing LNnT will also result in formation of significant amounts of LNFP-VI, but no LNFP-III or LNDFH-III.

For the production of more complex fucosylated HMOs, the genetically engineered cell described herein may comprise one or more further recombinant nucleic acids encoding one or more recombinant and/or heterologous glycosyltransferases capable of transferring a glycosyl residue from a glycosyl donor to an acceptor oligosaccharide. Preferably, the additional glycosyltransferase(s) enables the genetically engineered cell to synthesize LNnT from a precursor molecule, such as lactose or LNT-II. In embodiments, the genetically engineered cell disclosed herein, comprises one or more further recombinant nucleic acid encoding one or more recombinant and/or heterologous glycosyltransferase.

Preferably, the additional glycosyltransferase(s) enables the genetically engineered cell to synthesize LNT or LNnT from a precursor molecule, such as lactose or LNT-II. In embodiments, the genetically engineered cell disclosed herein, comprises one or more further recombinant nucleic acids encoding one or more heterologous glycosyltransferases.

The additional glycosyltransferase is preferably selected from the group consisting of, galactosyltransferases, glucosaminyltransferases, fucosyltransferases, N-acetylglucosaminyl transferases and sialyltransferases.

The fucosyltransferase in the genetically engineered cell disclosed herein is an α-1,3-fucosyltransferase. Preferably, the α-1,3-fucosyltransferase is capable of transferring a fucose unit onto the Glc moiety of lactose.

The α-1,3-fucosyltransferases disclosed herein may also be capable of transferring a fucose unit onto the GlcNAc and/or Glc moiety of LNT, LNnT, LNFP-II, LNFP-III, LNFP-V and/or LNFP-VI.

Where it is desired to produce more complex fucosylated HMOs using the fucosyltransferases described herein, additional glycosyltransferases may be needed to produce the desired acceptor molecule for the α-1,3-fucosyltransferase, to enable the production of more complex fucosylated HMOs. In embodiments, such glycosyltransferases may be introduced into the cell and are preferably a β-1,3-galactosyltransferase, or a β-1,4-galactosyltransferase, or a combination of a β-1,3-galactosyltransferase and a β-1,3-N-acetyl-glucosaminyl-transferases, or combination of a β-1,4-galactosyltransferase and a β-1,3-N-acetyl-glucosaminyl-transferase.

In one embodiment, the α-1,3-fucosyltransferases Osc1 is introduced into a genetically engineered cell wherein said cell further comprises a β-1,4-galactosyltransferase, or a β-1,3-galactosyltransferase, and preferably also further comprises a β-1,3-N-acetyl-glucosaminyl-transferase.

In one embodiment, the α-1,3-fucosyltransferases Bbac1 is introduced into a genetically engineered cell wherein said cell further comprises a β-1,4-galactosyltransferase or a β-1,3-galactosyltransferase, and preferably also further comprises a β-1,3-N-acetyl-glucosaminyl-transferase.

In one embodiment, the α-1,3-fucosyltransferases Murba1 is introduced into a genetically engineered cell wherein said cell further comprises a β-1,4-galactosyltransferase or a β-1,3-galactosyltransferase, and preferably also further comprises a β-1,3-N-acetyl-glucosaminyl-transferase.

In one embodiment, the α-1,3-fucosyltransferases Bacbac1 is introduced into a genetically engineered cell wherein said cell further comprises a β-1,4-galactosyltransferase or a β-1,3-galactosyltransferase, and preferably also further comprises a β-1,3-N-acetyl-glucosaminyl-transferase.

The β-1,3-galactosyltransferases can be obtained from a number of sources, e.g., the ga/TK gene from H. pylori (homologous to GenBank protein Accession BD182026.1), or the WbgO gene from E. coli 055:H7 (GenBank Accession WP_000582563.1), or the jhp0563 gene from H. pylori (GenBank Accession AEZ55696.1). The β-1,4-glycosyltransferases can be obtained from a number of sources, e.g. GaIT from Helicobacter pylori (homologous to GenBank protein Accession WP_001262061.1), or LgtB from Neisseria meningitidis MC58 (homologous to GenBank protein Accession AAF42257.1).

The β-1,3-N-acetylglucosaminyltransferases can be obtained from a number of sources, e.g., the IgtA genes from N. meningitidis (e.g., GenBank protein Accession ID's AAF42258.1, WP_002248149.1, or WP_033911473.1 or ELK60643.1), or β-1,3-N-acetylglucosaminyltransferases genes from N. gonorrhoeae (e.g., GenBank protein Accession No's ACF31229.1, or AAK70338.1), or β-1,3-N-acetylglucosaminyltransferases genes from Haemophilus ducreyi (e.g., GenBank protein Accession AAN05638.1), or β-1,3-N-acetylglucosaminyltransferases genes from Pasteurella multocida (e.g., GenBank protein Accession AAK02595.1), or β-1,3-N-acetylglucosaminyltransferases genes from Neisseria cinerea (e.g., GenBank protein Accession EEZ72046.1).

In one embodiment, the enzyme Osc1 is introduced into a genetically engineered cell which further comprises an alpha-2,3-sialyltransferase, wherein the cell is capable of producing FSL, such as a mixture of 3′SL, 3FL and FSL.

In one embodiment, the enzyme Bacbac1 is introduced into a genetically engineered cell which further comprises an alpha-2,3-sialyltransferase, wherein the cell is capable of producing FSL, such as a mixture of 3′SL, 3FL and FSL.

In one embodiment, the enzyme Murba1 is introduced into a genetically engineered cell which further comprises an alpha-2,3-sialyltransferase, wherein the cell is capable of producing FSL, such as a mixture of 3′SL, 3FL and FSL.

In one embodiment, the enzyme Bgall1 is introduced into a genetically engineered cell which further comprises an alpha-2,3-sialyltransferase, wherein the cell is capable of producing FSL, such as a mixture of 3′SL, 3FL and FSL.

The α-2,3-sialyltransferase can be obtained from a number of sources, e.g., Clari1 from Campylobacterlari (GenBank protein Accession No. EGK8106227.1), Neigon from Neisseria gonorrhoeae FA 1090 (GenBank protein Accession No. AAW89748.1), Poral from Pasteurella oralis (GenBank protein Accession No. WP_101774487.1) and PmN from Pasteurella GenBank protein Accession No. WP_005726268.1).

### Glycosyl-Donor—Nucleotide-Activated Sugar Pathways

When carrying out the method of this disclosure, preferably a glycosyltransferase mediated glycosylation reaction takes place in which an activated sugar nucleotide serves as glycosyl-donor. An activated sugar nucleotide generally has a phosphorylated glycosyl residue attached to a nucleoside. A specific glycosyl transferase enzyme accepts only a specific sugar nucleotide. Thus, preferably the following activated sugar nucleotides are involved in the glycosyl transfer: glucose-UDP-GlcNAc, UDP-galactose, UDP-glucose, UDP-N-acetylglucosamine (UDP-GlcNAc), UDP-N-acetylgalactosamine (UDP-galNAc) and CMP-N-acetylneuraminic acid.

The genetically engineered cell according to the present disclosure can comprise one or more pathways to produce a nucleotide-activated sugar selected from the group consisting of glucose-UDP-GlcNAc, GDP-fucose, UDP-galactose, UDP-glucose, UDP-N-acetylglucosamine, UDP-N-acetylgalactosamine and CMP-N-acetylneuraminic acid.

In one embodiment of the current disclosure, the genetically engineered cell is capable of producing one or more activated sugar nucleotides mentioned above by a de novo pathway. In this regard, an activated sugar nucleotide is made by the cell under the action of enzymes involved in the de novo biosynthetic pathway of that respective sugar nucleotide in a stepwise reaction sequence starting from a simple carbon source like glycerol, sucrose, fructose or glucose (for a review for monosaccharide metabolism see e.g. H. H. Freeze and A. D. Elbein: Chapter 4: Glycosylation precursors, in: Essentials of Glycobiology, 2nd edition (Eds. A. Varki et al.), Cold Spring Harbour Laboratory Press (2009)).

The enzymes involved in the de novo biosynthetic pathway of an activated sugar nucleotide can be naturally present in the cell or introduced into the cell by means of gene technology or recombinant DNA techniques, all of them are parts of the general knowledge of the skilled person.

In another embodiment, the genetically engineered cell can utilize salvaged monosaccharides for sugar nucleotide. In the salvage pathway, monosaccharides derived from degraded oligosaccharides are phosphorylated by kinases, and converted to nucleotide sugars by pyrophosphorylases. The enzymes involved in the procedure can be heterologous ones, or native ones of the host cell.

### Colanic Acid Gene Cluster

For the production of fucosylated HMOs, the de novo GDP-fucose pathway is important to ensure presence of sufficient GDP-fucose. The colanic acid gene cluster of Escherichia coli encodes selected enzymes involved in the de novo synthesis of GDP-fucose (gmd, wcaG, wcaH, wcal, manB, manC), whereas one or several of the genes downstream of GDP-L-fucose such as wcaJ, which are responsible for the production of the extracellular polysaccharide colanic acid, a major oligosaccharide of the bacterial cell wall, can be deleted to prevent conversion of GDP-fucose to colanic acid.

To secure sufficient amounts of GDP-fucose the promoter of the native colanic acid gene cluster may be exchanged with a stronger promoter, generating a recombinant colanic acid gene cluster, to drive additional production of GDP-fucose. Furthermore, an extra copy of the colanic acid gene cluster or selected genes thereof can be introduced in the genetically engineered cells as described in the examples.

In embodiments, the colanic acid gene cluster may be expressed from its native genomic locus. The expression may be actively modulated. The expression can be modulated by swapping the native promoter with a promoter of interest, and/or increasing the copy number of the colanic acid genes coding said protein(s) by expressing the gene cluster from another genomic locus than the native, or episomally expressing the colanic acid gene cluster or specific genes thereof.

In relation to the present disclosure, the term “native genomic locus”, in relation to the colanic acid gene cluster, relates to the original and natural position of the gene cluster in the genome of the genetically engineered cell.

The de novo GDP-fucose pathway genes responsible for the formation of GDP-fucose comprises or consists of the following genes:
i) manA which encodes the protein mannose-6 phosphate isomerase (EC 5.3.1.8, UniProt accession nr. P00946), which facilitates the interconversion of fructose 6-phosphate (F6P) and mannose-6-phosphate;
ii) manB which encodes the protein phosphomannomutase (EC 5.4.2.8, UniProt accession nr P24175), which is involved in the biosynthesis of GDP-mannose by catalyzing conversion mannose-6-phosphate into mannose-1-phosphate;
iii) manC which encodes the protein mannose-1-phosphate guanylyltransferase guanylyltransferase (EC:2.7.7.13, UniProt accession nr P24174), which is involved in the biosynthesis of GDP-mannose through synthesis of GDP-mannose from GTP and α-D-mannose-1-phosphate;
iv) gmd which encodes the protein GDP-mannose-4,6-dehydratase (UniProt accession nr POAC88), which catalyzes the conversion of GDP-mannose to GDP-4-dehydro-6-deoxy-D-mannose;
v) wcaG (fc/) which encodes the protein GDP-L-fucose synthase (EC 1.1.1.271, UniProt accession nr P32055) which catalyses the two-step NADP-dependent conversion of GDP-4-dehydro-6-deoxy-D-mannose to GDP-fucose.

Accordingly, it is preferred that the genetically engineered cell, when producing one or more fucosylated heterologous products, overexpresses either the entire colonic acid gene cluster and/or one or more genes of the de novo GDP-fucose pathway selected from the group consisting of manA, manB, manC, gmd and wcaG.

### Lactose Permease

Lactose permease is a membrane protein which is a member of the major facilitator superfamily and can be classified as a symporter, which uses the proton gradient towards the cell to transport β-galactosides such as lactose in the same direction into the cell. In oligosaccharide-production, especially in the production of human milk oligosaccharides (HMOs), lactose is often the initial substrate being decorated to produce any HMO of interest in a bioconversion that happens in the cell interior. Thus, in the production of HMOs, there is a desire to be able to import lactose into the cell, e.g., by expression and/or overexpression of a lactose permease such as lacY of E. coli.

In embodiments, the lactose permease is as shown in SEQ ID NO: 12, or a functional homologue thereof having an amino acid sequence which is at least 80% identical, such as at least 85%, 90% or 95% identical to SEQ ID NO: 12.

In embodiments, the expression of the lactose permease is regulated by a promoter according to the present disclosure.

β-galactosidase

A host cell suitable for HMO production, e.g., E. coli, may comprise an endogenous β-galactosidase gene or an exogenous β-galactosidase gene, e.g., E. coli comprises an endogenous lacZ gene (e.g., GenBank Accession Number V00296 (GI:41901)). For the purposes of the disclosure, when producing an HMO it is preferred that, the genetically engineered cell does not express a functional β-galactosidase to avoid the degradation of lactose, if lactose is used as the initial substrate for producing the complex fucosylated HMO. In embodiments the gene may be inactivated by a complete or partial deletion of the corresponding nucleic acid sequence from the bacterial genome, or the gene sequence is mutated in the way that it is not transcribed, or, if transcribed, the transcript is not translated or if translated to a protein (i.e., β-galactosidase), the protein does not have the corresponding enzymatic activity. In this way the HMO-producing bacterium accumulates an increased intracellular lactose pool which is beneficial for the production of HMOs.

### Transporter Proteins

The oligosaccharide product, such as the HMO produced by the cell, can be accumulated both in the intra- and the extracellular matrix. The product can be transported to the supernatant in a passive way, i.e., it diffuses outside across the cell membrane. The more complex HMO products may remain in the cell, which is likely to eventually impair cellular growth, thereby affecting the possible total yield of the product from a single fermentation. The HMO transport can be facilitated by major facilitator superfamily transporter proteins that promote the effluence of sugar derivatives from the cell to the supernatant. The transporter can be present exogenously or endogenously and is overexpressed under the conditions of the fermentation to enhance the export of the oligosaccharide derivative (HMO) produced. The specificity towards the sugar moiety of the product to be secreted can be altered by mutation by means of known recombinant DNA techniques.

Thus, the genetically engineered cell according to the present disclosure can further comprise a nucleic acid sequence encoding a transporter protein capable of exporting the fucosylated human milk oligosaccharide product or products, such as transporter protein can for example be a member of the major facilitator superfamily transport proteins.

In the resent years, several new and efficient major facilitator superfamily transporter proteins have been identified, each having specificity for different recombinantly produced HMOs and development of recombinant cells expressing said proteins are advantageous for high scale industrial HMO manufacturing.

In one embodiment the genetically engineered cell of the present disclosure preferably expresses a heterologous Major Facilitator Superfamily (MFS) transporter protein.

The term “MFS transporter” in the present context means a protein that facilitates transport of an oligosaccharide, preferably an HMO, through or across a cell membrane, from the cell cytosol to the cell periplasm and/or medium.

In one embodiment the genetically engineered cell comprises an MFS transporter protein selected from the group consisting of MFS transporter proteins comprising or consisting of an amino acid sequence according to the GenBank accession WP_060448169.1, WP_092672081.1, WP_087817556.1, and WP_048785139.1, or a functional homologue of any one of said MFS transporters, having an amino acid sequence which is at least 80%, such as at least 90%, such as at least 95%, such as at least 99%, such as 100% identical to the amino acid sequence of GenBank accession WP_060448169.1, WP_092672081.1, WP_087817556.1, or WP_048785139.1.

In one embodiment the genetically engineered cell comprises an MFS transporter protein with an amino acid sequence corresponding the GenBank accession WP_060448169.1 or a functional homologue thereof, having an amino acid sequence which is at least 80%, such as at least 90%, such as at least 95%, such as at least 99%, such as 100% identical to the amino acid sequence of GenBank accession WP_060448169.1.

In one embodiment the genetically engineered cell comprises an MFS transporter protein with an amino acid sequence corresponding the GenBank accession WP_092672081.1, or a functional homologue thereof, having an amino acid sequence which is at least 80%, such as at least 90%, such as at least 95%, such as at least 99%, such as 100% identical to the amino acid sequence of GenBank accession WP_092672081.1.

In one embodiment the genetically engineered cell comprises an MFS transporter protein with an amino acid sequence corresponding the GenBank accession WP_087817556.1, or a functional homologue thereof, having an amino acid sequence which is at least 80%, such as at least 90%, such as at least 95%, such as at least 99%, such as 100% identical to the amino acid sequence of GenBank accession WP_087817556.1.

In one embodiment the genetically engineered cell comprises an MFS transporter protein with an amino acid sequence corresponding the GenBank accession WP_048785139.1, or a functional homologue thereof, having an amino acid sequence which is at least 80%, such as at least 90%, such as at least 95%, such as at least 99%, such as 100% identical to the amino acid sequence of GenBank accession WP_048785139.1.

### The Genetically Engineered Cell

In the present context, the terms “a genetically engineered cell” and “a genetically modified cell” are used interchangeably. As used herein “a genetically engineered cell” is a host cell whose genetic material has been altered by human intervention using a genetic engineering technique, such a technique is e.g., but not limited to transformation or transfection e.g., with a heterologous and/or recombinant polynucleotide sequence, Crisper/Cas editing and/or random mutagenesis. In one embodiment the genetically engineered cell has been transformed or transfected with a recombinant nucleic acid sequence.

The genetic modifications can e.g., be selected from inclusion of glycosyltransferases, and/or metabolic pathway engineering, deletion of repressors or undesired enzymes and inclusion of transporters as described in the above sections, which the skilled person will know how to combine into a genetically engineered cell capable of producing 3FL.

In embodiments, the genetically engineered cell capable of producing 3FL, comprises a recombinant nucleic acid sequence encoding a fucosyltransferase with α-1,3-fucosyltransferase activity, selected from the group consisting of, a) Osc1, comprising or consisting of the amino acid sequence of SEQ ID NO: 1, or a functional homologue thereof with an amino acid sequence that is at least 80% identical to SEQ ID NO: 1, b) Bbac1 comprising or consisting of the amino acid sequence of SEQ ID NO: 2, or a functional homologue thereof with an amino acid sequence that is at least 80% identical to SEQ ID NO: 2, c) Murba1 comprising or consisting of the amino acid sequence of SEQ ID NO: 3, or a functional homologue thereof with an amino acid sequence that is at least 80% identical to SEQ ID NO: 3 and d) Bacbac1 comprising or consisting of the amino acid sequence of SEQ ID NO: 4, or a functional homologue thereof with an amino acid sequence that is at least 80% identical to SEQ ID NO: 4.

Preferably, the α-1,3-fucosyltransferase disclosed herein is capable of fucosylating lactose at the Glu moiety with an α-1,3 linkage.

In embodiments, the genetically engineered cell capable of producing 3FL, comprises a recombinant nucleic acid sequence encoding the α-1,3-fucosyltransferase Osc1, comprising or consisting of the amino acid sequence of SEQ ID NO: 1, or a functional homologue thereof with an amino acid sequence that is at least 80% identical to SEQ ID NO: 1.

In embodiments, the genetically engineered cell capable of producing 3FL, comprises a recombinant nucleic acid sequence encoding the α-1,3-fucosyltransferase Bbac1 comprising or consisting of the amino acid sequence of SEQ ID NO: 2, or a functional homologue thereof with an amino acid sequence that is at least 80% identical to SEQ ID NO: 2.

In embodiments, the genetically engineered cell capable of producing 3FL, comprises a recombinant nucleic acid sequence encoding the α-1,3-fucosyltransferase Murba1 comprising or consisting of the amino acid sequence of SEQ ID NO: 3, or a functional homologue thereof with an amino acid sequence that is at least 80% identical to SEQ ID NO: 3.

In embodiments, the genetically engineered cell capable of producing 3FL, comprises a recombinant nucleic acid sequence encoding the α-1,3-fucosyltransferase Bacbac1 comprising or consisting of the amino acid sequence of SEQ ID NO: 4, or a functional homologue thereof with an amino acid sequence that is at least 80% identical to SEQ ID NO: 4.

The genetically engineered cell described herein preferably expresses genes encoding key enzymes for the biosynthesis of fucosylated HMOs. Preferably the genetically engineered cell disclosed herein expresses genes encoding key enzymes for the biosynthesis of fucosylated HMOs.

In some embodiments, a genetically engineered cell disclosed herein further expresses the de novo GDP-fucose pathway genes responsible for the formation of GDP-fucose manA, manB, manC, gmd and wcaG. It may be advantageous to overexpress one or more of these genes and/or to upregulate the colanic acid gene cluster (CA), including the genes gmd, wcaG, wcaH, wcal, manC and manB from E. Coli, through introduction of a nucleic acid construct encoding the CA as shown in SEQ ID NO: 11, allowing for formation of GDP-fucose, which enables the cell to produce a higher level of fucosylated oligosaccharides from lactose. Depending on the intended use of substrate and desirable final product, one or more additional glycosyltransferases and pathways for producing nucleotide-activated sugars, such as glucose-UDP-GlcNAc, CMP-N-acetylneuraminic acid, UDP-galactose, UDP-glucose, UDP-N-acetylglucosamine, UDP-N-acetylgalactosamine and/or CMP-N-acetylneuraminic acid can also be present in the genetically engineered cell.

### Host Cells

In embodiments, the engineered cell is a microorganism. The genetically engineered cell is preferably a microbial cell, such as a prokaryotic cell or eukaryotic cell. Appropriate microbial cells that may function as a host cell include bacterial cells, archaebacterial cells, algae cells and fungal cells.

The genetically engineered cell may be e.g., a bacterial or yeast cell. In one preferred embodiment, the genetically engineered cell is a bacterial cell.

Regarding the bacterial host cells, there are, in principle, no limitations; they may be eubacteria (gram-positive or gram-negative) or archaebacteria, as long as they allow genetic manipulation for insertion of a gene of interest and can be cultivated on a manufacturing scale. Preferably, the host cell has the property to allow cultivation to high cell densities. Non-limiting examples of bacterial host cells that are suitable for recombinant industrial production of an HMO(s) according to the disclosure could be member of the Enterobacterales order, preferably of the genus Escherichia, more preferably of the species E. coli. Other examples of suitable host cell are Erwinia herbicola (Pantoea agglomerans), Citrobacter freundii, Campylobacter sp, Pantoea citrea, Pectobacterium carotovorum, or Xanthomonas campestris. Bacteria of the genus Bacillus may also be used, including Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, and Bacillus circulans. Similarly, bacteria of the genera Lactobacillus and Lactococcus may be engineered using the methods of this disclosure, including but not limited to Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillus jensenii, and Lactococcus lactis. Streptococcus thermophiles and Proprionibacterium freudenreichii are also suitable bacterial species. Also included as useful species are strains, engineered as described here, from the genera Enterococcus (e.g., Enterococcus faecium and Enterococcus thermophiles), Bifidobacterium (e.g., Bifidobacterium longum, Bifidobacterium infantis, and Bifidobacterium bifidum), Sporolactobacillus spp., Micromomospora spp., Micrococcus spp., Rhodococcus spp., and Pseudomonas (e.g., Pseudomonas fluorescens and Pseudomonas aeruginosa).

Non-limiting examples of fungal host cells that are suitable for recombinant industrial production of a heterologous product are e.g., yeast cells, such as Komagataella, Kluyveromyces, Yarrowia, Pichia, Saccaromyces, Schizosaccharomyces or Hansenula or from a filamentous fungus of the genera Aspargillus, Fusarium or Thricoderma.

In one or more exemplary embodiments, the genetically engineered cell is selected from the group consisting of Escherichia sp., Bacillus sp., lactobacillus sp., Corynebacterium sp. and Campylobacter sp.

In one or more exemplary embodiments, the genetically engineered cell is selected from the group consisting of Escherichia coli, Bacillus subtilis, lactobacillus lactis, Corynebacterium glutamicum, Yarrowia lipolytica, Pichia pastoris, and Saccharomyces cerevisiae.

In one or more exemplary embodiments, the genetically engineered cell is B. subtilis.

In one or more exemplary embodiments, the genetically engineered cell is S. Cerevisiae or P pastoris.

In one or more exemplary embodiments, the genetically engineered cell is Escherichia coli.

In one or more exemplary embodiments, the disclosure relates to a genetically engineered cell, wherein the cell is derived from the E. coli K-12 strain or DE3.

### A Recombinant Nucleic Acid Sequence

The present disclosure relates to a genetically engineered cell comprising a recombinant nucleic acid sequence encoding an α-1,3-fucosyltransferase, such as an enzyme selected from the group consisting of Osc1, Bbac1, Murba1 and Bacbac1, wherein said cell produces Human Milk Oligosaccharides (HMO). In particular, a fucosylated HMO, and preferably the HMO 3FL is produced.

In the present context, the term “recombinant nucleic acid sequence”, “recombinant gene/nucleic acid/nucleotide sequence/DNA encoding” or “coding nucleic acid sequence” is used interchangeably and intended to mean an artificial nucleic acid sequence (i.e. produced in vitro using standard laboratory methods for making nucleic acid sequences) that comprises a set of consecutive, non-overlapping triplets (codons) which is transcribed into mRNA and translated into a protein when under the control of the appropriate control sequences, i.e., a promoter sequence.

The boundaries of the coding sequence are generally determined by a ribosome binding site located just upstream of the open reading frame at the 5′end of the mRNA, a transcriptional start codon (AUG, GUG or UUG), and a translational stop codon (UAA, UGA or UAG). A coding sequence can include, but is not limited to, genomic DNA, cDNA, synthetic, and recombinant nucleic acid sequences.

The term “nucleic acid” includes RNA, DNA and cDNA molecules. It is understood that, as a result of the degeneracy of the genetic code, a multitude of nucleic acid sequences encoding a given protein may be produced.

The recombinant nucleic acid sequence may be a coding DNA sequence e.g., a gene, or non-coding DNA sequence e.g., a regulatory DNA, such as a promoter sequence or other non-coding regulatory sequences.

The recombinant nucleic acid sequence may in addition be heterologous. As used herein “heterologous” refers to a polypeptide, amino acid sequence, nucleic acid sequence or nucleotide sequence that is foreign to a cell or organism, i.e., to a polypeptide, amino acid sequence, nucleic acid molecule or nucleotide sequence that does not naturally occurs in said cell or organism.

The disclosure also relates to a nucleic acid construct comprising a coding nucleic sequence, i.e. recombinant DNA sequence of a gene of interest, e.g., an α-1,3-fucosyltransferase gene, and a non-coding regulatory DNA sequence, e.g., a promoter DNA sequence, e.g., a recombinant promoter sequence derived from the promoter sequence of the lac operon or the glp operon, or a promoter sequence derived from another genomic promoter DNA sequence, or a synthetic promoter sequence, wherein the coding and promoter sequences are operably linked.

The term “operably linked” refers to a functional relationship between two or more nucleic acid (e.g., DNA) segments. It refers to the functional relationship of a transcriptional regulatory sequence to a transcribed sequence. E.g., a promoter sequence is operably linked to a coding sequence if it stimulates or modulates the transcription of the coding sequence in an appropriate host cell or other expression system.

Generally, promoter sequences that are operably linked to a transcribed sequence are physically contiguous to the transcribed sequence, i.e., they are cis-acting.

In one exemplified embodiment, the nucleic acid construct of the disclosure may be a part of the vector DNA, in another embodiment, the construct it is an expression cassette/cartridge that is integrated in the genome of a host cell.

Accordingly, the term “nucleic acid construct” means an artificially constructed segment of nucleic acids, in particular a DNA segment, which is intended to be inserted into a target cell, e.g., a bacterial cell, to modify expression of a gene of the genome or expression of a gene/coding DNA sequence which may be included in the construct. Thus, in embodiments, the present disclosure relates to a nucleic acid construct comprising a recombinant nucleic acid sequence encoding an α-1,3-fucosyltransferase, wherein said recombinant nucleic acid sequence is selected from the group consisting of nucleic acid sequences encoding Osc1, Bbac1, Murba1 and Bacbac1, such as a nucleic acid sequence according to SEQ ID NO: 6, 7, 8 and 9, or functional variants thereof.

The genetically engineered cell according to the present disclosure may also comprise multiple copies of the recombinant nucleic acid sequence encoding an α-1,3-fucosyltransferase. Enhancing the copy number of the α-1,3-fucosyltransferase may be used to further enhance the 3FL production.

Accordingly, in embodiments, the genetically engineered cell disclosed herein comprises one, two, three or more genomic copies of the recombinant nucleic acid sequence encoding the glycosyltransferase selected from the group consisting of a) Osc1, comprising or consisting of the amino acid sequence of SEQ ID NO: 1, or a functional homologue thereof with an amino acid sequence that is at least 80% identical to SEQ ID NO: 1, b) Bbac1 comprising or consisting of the amino acid sequence of SEQ ID NO: 2, or a functional homologue thereof with an amino acid sequence that is at least 80% identical to SEQ ID NO: 2, c) Murba1 comprising or consisting of the amino acid sequence of SEQ ID NO: 3, or a functional homologue thereof with an amino acid sequence that is at least 80% identical to SEQ ID NO: 3 and D) Bacbac1 comprising or consisting of the amino acid sequence of SEQ ID NO: 4, or a functional homologue thereof with an amino acid sequence that is at least 80% identical to SEQ ID NO: 4.

In further embodiments, the recombinant nucleic acid sequence encoding the glycosyltransferase selected from the group consisting of a) Osc1, comprising or consisting of the amino acid sequence of SEQ ID NO: 1, or a functional homologue thereof with an amino acid sequence that is at least 80% identical to SEQ ID NO: 1, b) Bbac1 comprising or consisting of the amino acid sequence of SEQ ID NO: 2, or a functional homologue thereof with an amino acid sequence that is at least 80% identical to SEQ ID NO: 2, c) Murba1 comprising or consisting of the amino acid sequence of SEQ ID NO: 3, or a functional homologue thereof with an amino acid sequence that is at least 80% identical to SEQ ID NO: 3 and d) Bacbac1 comprising or consisting of the amino acid sequence of SEQ ID NO: 4, or a functional homologue thereof with an amino acid sequence that is at least 80% identical to SEQ ID NO: 4, wherein said recombinant nucleic acid sequence is encoded on a plasmid. In additional embodiments, the plasmid is a high copy number plasmid, preferably, a pUC57 or pBB-B9 plasmid.

One embodiment relates to a nucleic acid construct comprising a recombinant nucleic acid sequence encoding an α-1,3-fucosyltransferase, wherein said recombinant nucleic acid sequence is selected from the group consisting of a) Osc1, comprising or consisting of the nucleic acid sequence of SEQ ID NO: 6, or a functional homologue thereof with an nucleic acid sequence that is at least 80% identical to SEQ ID NO: 6, b) Bbac1 comprising or consisting of the nucleic acid sequence of SEQ ID NO: 7, or a functional homologue thereof with an nucleic acid sequence that is at least 80% identical to SEQ ID NO: 7, c) Murba1 comprising or consisting of the nucleic acid sequence of SEQ ID NO: 8, or a functional homologue thereof with an nucleic acid sequence that is at least 80% identical to SEQ ID NO: 8 and D) Bacbac1 comprising or consisting of the nucleic acid sequence of SEQ ID NO: 9, or a functional homologue thereof with an nucleic acid sequence that is at least 80% identical to SEQ ID NO: 9.

Preferably, the α-1,3-fucosyltransferase encoding sequence is under the control of a promoter sequence selected from promotor sequences with a nucleic acid sequence as identified in Table 2.

TABLE 2
Selected promoter sequences

| | % Activity | | | Seq ID |
|---|---|---|---|---|
| Promoter name | relative to PglpF* | Strength | Reference | in appl. |
| PmglB_70UTR_SD8 | 291% | high | WO2020255054 | 13 |
| PmglB_70UTR_SD10 | 233-281% | high | WO2020255054 | 14 |
| PmglB_54UTR | 197% | high | WO2020255054 | 15 |
| Plac_70UTR | 182-220% | high | WO2019123324 | 16 |
| PmglB_70UTR_SD9 | 180-226% | high | WO2020255054 | 17 |
| PmglB_70UTR_SD4 | 153%-353% | high | WO2020255054 | 18 |
| PmglB_70UTR_SD5 | 146-152% | high | WO2020255054 | 19 |
| PglpF_SD4 | 140-161% | high | WO2019123324 | 20 |
| PmglB_70UTR_SD7 | 127-173% | high | WO2019123324 | 21 |
| PmglB_70UTR | 124-234% | high | WO2020255054 | 22 |
| PglpA_70UTR | 102-179% | high | WO2019123324 | 23 |
| PglpT_70UTR | 102-240% | high | WO2019123324 | 24 |
| PglpF | 100% | high | WO2019123324 | 25 |
| PglpF_SD10 | 88-96% | high | WO2019123324 | 26 |
| PglpF_SD5 | 82-91% | high | WO2019123324 | 27 |
| PglpF_SD8 | 81-82% | high | WO2019123324 | 28 |
| PmglB_16UTR | 78-171% | high | WO2019123324 | 29 |
| PglpF_SD9 | 73-93% | middle | WO2019123324 | 30 |
| PglpF_SD7 | 47-57% | middle | WO2019123324 | 31 |
| PglpF_SD6 | 46-47% | middle | WO2019123324 | 32 |
| PglpA_16UTR | 38-64% | middle | WO2019123324 | 33 |
| Plac_wt* | 15-28% | low | WO2019123324 | 34 |
| PglpF_SD3 | 9% | low | WO2019123324 | 35 |
| PglpF_SD1 | 5% | low | WO2019123324 | 36 |

*The promoter activity is assessed in the LacZ assay described below with the PglpF promoter run as positive reference in the same assay. To compare across assays the activity is calculated relative to the PglpF promoter, a range indicates results from multiple assays.

The promoter may be of heterologous origin, native to the genetically engineered cell or it may be a recombinant promoter, combining heterologous and/or native elements.

One way to increase the production of a product may be to regulate the production of the desired enzyme activity used to produce the product, such as the glycosyltransferases or enzymes involved in the biosynthetic pathway of the glycosyl donor.

Increasing the promoter strength driving the expression of the desired enzyme may be one way of doing this. The strength of a promoter can be assessed using a lacZ enzyme assay where β-galactosidase activity is assayed as described previously (see e.g., Miller J. H. Experiments in molecular genetics, Cold spring Harbor Laboratory Press, NY, 1972). Briefly the cells are diluted in Z-buffer and permeabilized with sodium dodecyl sulfate (0.1%) and chloroform. The LacZ assay is performed at 30° C. Samples are preheated, the assay initiated by addition of 200 μl ortho-nitro-phenyl-β-galactosidase (4 mg/ml) and stopped by addition of 500 μl of 1 M Na₂CO₃ when the sample had turned slightly yellow. The release of ortho-nitrophenol is subsequently determined as the change in optical density at 420 nm. The specific activities are reported in Miller Units (MU) [A420/(min*ml*A600)]. A regulatory element with an activity above 10,000 MU is considered strong and a regulatory element with an activity below 3,000 MU is considered weak, what is in between has intermediate strength. An example of a strong regulatory element is the PglpF promoter with an activity of approximately 14.000 MU and an example of a weak promoter is Plac which when induced with IPTG has an activity of approximately 2300 MU. In preferred embodiments, the expression of said nucleic acid sequences are under control of a strong promoter selected from the group consisting of SEQ ID NOs 13, 14, 15, 16, 17, 18, 19, 20, 21, 22, 23 and 24.

In embodiments the expression of said nucleic acid sequences disclosed herein is under control of a PglpF (SEQ ID NO: 25) or Plac (SEQ ID NO: 34) promoter or PmglB_UTR70 (SEQ ID NO: 22) or PglpA_70UTR (SEQ ID NO: 23) or PglpT_70UTR (SEQ ID NO: 24) or variants thereof such as promoters identified in Table 2, in particular the PglpF_SD4 variant of SEQ ID NO: 20 or Plac_70UTR variant of SEQ ID NO: 16, or Pmg|B_70UTR variants of SEQ ID NO: 13, 14, 17, 18, 19, 21 and 22. Further suitable variants of PglpF, PglpA_70UTR, PglpT_70UTR and Pmg|B_70UTR promoter sequences are described in or WO2019/123324 and WO2020/255054 respectively (hereby incorporated by reference).

In preferred embodiments, the recombinant nucleic acid sequences individually are under the control of one or more promoters selected from the group consisting of PglpF, Plac, Pmg|B_70UTR, PglpA_70UTR and PglpT_70UTR (SEQ ID NOs: 25, 34, 22, 23 and 24, respectively) and variants thereof.

Integration of the nucleic acid construct of interest comprised in the construct (expression cassette) into the bacterial genome can be achieved by conventional methods, e.g. by using linear cartridges that contain flanking sequences homologous to a specific site on the chromosome, as described for the attTn7-site (Waddell C. S. and Craig N. L., Genes Dev. (1988) February; 2(2):137-49.); methods for genomic integration of nucleic acid sequences in which recombination is mediated by the Red recombinase function of the phage λ or the RecE/RecT recombinase function of the Rac prophage (Murphy, J Bacteriol. (1998); 180(8):2063-7; Zhang et al., Nature Genetics (1998) 20: 123-128 Muyrers et al., EMBO Rep. (2000) 1(3): 239-243); methods based on Red/ET recombination (Wenzel et al., Chem Biol. (2005), 12(3):349-56.; Vetcher et al., Appl Environ Microbiol. (2005); 71(4):1829-35); or positive clones, i.e., clones that carry the expression cassette, can be selected e.g., by means of a marker gene, or loss or gain of gene function.

In one or more exemplary embodiments, the present disclosure relates to one or more recombinant nucleic acid sequences as illustrated in SEQ ID NOs 6, 7, 8 and 9.

In particular, the present disclosure relates to one or more of a recombinant nucleic acid sequence and/or to a functional homologue thereof having a sequence which is at least 70% identical to SEQ ID NOs: 6, 7, 8 and 9 [nucleic acid encoding Osc1, Bbac1, Murba1 and bacbac1, respectively], such as at least 75% identical, at least 80% identical, at least 85% identical, at least 90% identical, at least, at least 95% identical, at least 98% identical, or 100% identical.

### Sequence Identity

The term “sequence identity” as used herein describes the relatedness between two amino acid sequences or between two nucleotide sequences, i.e., a candidate sequence (e.g., a sequence of the invention) and a reference sequence (such as a prior art sequence) based on their pairwise alignment. For purposes of the present invention, the sequence identity between two amino acid sequences is determined using the Needleman-Wunsch algorithm (Needleman and Wunsch, 1970, J. Mo/. Biol. 48: 443-453) as implemented in the Needle program of the EMBOSS package (EMBOSS: The European Molecular Biology Open Software Suite, Rice et al., 2000, Trends Genet. 16: 276-277,), preferably version 5.0.0 or later (available at https://www.ebi.ac.uk/Tools/psa/emboss needle/). The parameters used are gap open penalty of 10, gap extension penalty of 0.5, and the EBLOSUM62 (EMBOSS version of 30 BLOSUM62) substitution matrix. The output of Needle labelled “identity” (obtained using the -nobrief option) is used as the percent identity. Generally sequence identity may be calculated as follows: (Identical Residues×100)/(Length of Aligned region).

For purposes of the present invention, the sequence identity between two nucleotide sequences is determined using the Needleman-Wunsch algorithm (Needleman and Wunsch, 1 970, supra) as implemented in the Needle program of the EMBOSS package (EMBOSS: The European Molecular Biology Open Software Suite, Rice et al., 2000, Trends Genet. 16: 276-277), 10 preferably version 5.0.0 or later. The parameters used are gap open penalty of 10, gap extension penalty of 0.5, and the DNAFULL (EMBOSS version of NCBI NUC4.4) substitution matrix. The output of Needle labelled “identity” (obtained using the -nobrief option) is used as the percent identity. Generally sequence identity may be calculated as follows: (Identical Deoxyribonucleotides×100)/(Length of Aligned region).

### Functional Homologue

A functional homologue or functional variant of a protein/nucleic acid sequence as described herein is a protein/nucleic acid sequence with alterations in the genetic code, which retain its original functionality. A functional homologue may be obtained by mutagenesis or may be natural occurring variants from the same or other species. The functional homologue should have a remaining functionality of at least 50%, such as at least 60%, 70%, 80%, 90% or 100% compared to the functionality of the protein/nucleic acid sequence.

A functional homologue of any one of the disclosed amino acid or nucleic acid sequences can also have a higher functionality. A functional homologue of any one of the amino acid sequences shown in table 1 or a recombinant nucleic acid encoding any one of the sequences of SEQ ID NO: 6, 7, 8 and 9, should ideally be able to participate in the production of fucosylated HMOs, in terms of increased HMO yield, export of HMO product out of the cell or import of substrate for the HMO production, such as a acceptor oligosaccharide of at least three monosaccharide units, improved purity/by-product formation, reduction in biomass formation, viability of the genetically engineered cell, robustness of the genetically engineered cell according to the disclosure, or reduction in consumables needed for the production.

### Use of a Genetically Engineered Cell or Enzyme

The disclosure also relates to any commercial use of the enzyme(s), genetically engineered cell(s) or the nucleic acid construct(s) disclosed herein, such as, but not limited to, in a method for producing one or more fucosylated human milk oligosaccharide (HMO), preferably, 3FL.

Accordingly, the present disclosure also relates to the use of an α-1,3-fucosyltransferase in production of a fucosylated product, wherein the α-1,3-fucosyltransferase is selected from the group consisting of a) Osc1, comprising or consisting of the amino acid sequence of SEQ ID NO: 1, or a functional homologue thereof with an amino acid sequence that is at least 80% identical to SEQ ID NO: 1, b) Bbac1 comprising or consisting of the amino acid sequence of SEQ ID NO: 2, or a functional homologue thereof with an amino acid sequence that is at least 80% identical to SEQ ID NO: 2, c) Murba1 comprising or consisting of the amino acid sequence of SEQ ID NO: 3, or a functional homologue thereof with an amino acid sequence that is at least 80% identical to SEQ ID NO: 3 and d) Bacbac1 comprising or consisting of the amino acid sequence of SEQ ID NO: 4, or a functional homologue thereof with an amino acid sequence that is at least 80% identical to SEQ ID NO: 4. In preferred embodiments, the fucosylated product is 3FL.

In embodiments, the α-1,3-fucosyltransferase disclosed herein is also used in the manufacturing of a fucosylated product, wherein the fucosylated product is one or more fucosylated oligosaccharides, such as one or more HMOs, preferably, 3FL, most preferred 3FL is the only HMO manufactured using the α-1,3-fucosyltransferase disclosed herein.

In an exemplified embodiment, the genetically engineered cell and/or the nucleic acid construct described herein is used in the manufacturing of HMOs. Preferably, in the manufacturing of 3FL.

In embodiments, the α-1,3-fucosyltransferase disclosed herein are also used in the manufacturing of a fucosylated product, wherein the fucosylated product is one or more fucosylated oligosaccharides, such as one or more HMOs, preferably, a mixture of HMOs.

Production of these HMO's may require the presence of two or more glycosyltransferase activities.

### A Method for Producing Fucosylated Human Milk Oligosaccharides (HMOs)

The present disclosure also relates to a method for producing one or more fucosylated human milk oligosaccharide (HMO), preferably 3FL said method comprises culturing a genetically engineered cell according to the present disclosure.

An aspect of the present disclosure is a method for producing 3FL, said method comprising the steps of
a. cultivating a genetically engineered cell disclosed herein; and
b. culturing the cell according to (a) in a suitable cell culture medium to produce said one or more fucosylated HMOs, and
c. optionally, purifying 3FL from the culture medium and/or biomass.

The present disclosure thus relates to a method for producing one or more fucosylated human milk oligosaccharide (HMO), said method comprising culturing a genetically engineered cell, said cell comprising:
a recombinant nucleic acid sequence encoding an α-1,3-fucosyltransferase, wherein said enzyme is selected from the group consisting of:
   a. Osc1, comprising or consisting of the amino acid sequence of SEQ ID NO: 1, or a functional homologue thereof with an amino acid sequence that is at least 80% identical to SEQ ID NO: 1,
   b. bac1 comprising or consisting of the amino acid sequence of SEQ ID NO: 2, or a functional homologue thereof with an amino acid sequence that is at least 80% identical to SEQ ID NO: 2,
   c. Murba1 comprising or consisting of the amino acid sequence of SEQ ID NO: 3, or a functional homologue thereof with an amino acid sequence that is at least 80% identical to SEQ ID NO: 3 and
   d. Bacbac1 comprising or consisting of the amino acid sequence of SEQ ID NO: 4, or a functional homologue thereof with an amino acid sequence that is at least 80% identical to SEQ ID NO: 4,
wherein said method produces a fucosylated HMO, preferably 3FL, preferably only 3FL.

In embodiments the genetically engineered cell is cultured in a suitable medium providing a suitable carbon source, and in the presence of lactose as the initial substrate. In further embodiments one or more HMOs selected form the groups consisting of 3FL, LNFP-II, LNFP-III, LNFP-V, LNFP-VI, LNDFH-II LNDFH-III, LNnT, LNT-II and pLNnH are produced by the method of the disclosure.

In addition to the α-1,3-fucosyltransferase it is understood that the genetically engineered cell used in the method for producing a fucosylated HMO may contain additional modifications as described herein.

The method disclosed herein comprises providing a glycosyl donor, which is synthesized separately by one or more genetically engineered cells and/or is exogenously added to the culture medium from an alternative source. Preferably, the glucosyl donor is produced by an endogenous or recombinant de novo pathway in the genetically engineered cell.

One embodiment disclosed herein, further comprises providing an acceptor saccharide as substrate for the HMO formation, the acceptor saccharide comprising at least two monosaccharide units, which is exogenously added to the culture medium and/or has been produced by a separate microbial fermentation.

In one embodiment, the method disclosed herein comprises providing an acceptor saccharide comprising at least two monosaccharide units, which is exogenously added to the culture medium and/or has been produced by a separate microbial fermentation and which is selected form lactose, LNT-II and LNnT. In a preferred embodiment the substrate for HMO formation is lactose which is fed to the culture during the fermentation of the genetically engineered cell.

The fucosylated human milk oligosaccharide (HMO) is retrieved from the culture, either from the culture medium and/or the genetically engineered cell.

Culturing/Fermenting Culturing, cultivation, fermenting or fermentation (used interchangeably herein) in a controlled bioreactor typically comprises (a) a first phase of exponential cell growth in a culture medium ensured by a carbon-source, and (b) a second phase of cell growth in a culture medium run under carbon limitation, where the carbon-source is added continuously together with the acceptor oligosaccharide, such as lactose, allowing formation of the HMO product in this phase. By carbon (sugar) limitation is meant the stage in the fermentation where the growth rate is kinetically controlled by the concentration of the carbon source (sugar) in the culture broth, which in turn is determined by the rate of carbon addition (sugar feed-rate) to the fermenter.

The terms “manufacturing” or “manufacturing scale” or “large-scale production” or “large-scale fermentation”, are used interchangeably and in the meaning of the disclosure defines a fermentation with a minimum volume of 100 L, such as 1000 L, such as 10.000 L, such as 100.000 L, such as 200.000 L culture broth. Usually, a “manufacturing scale” process is defined by being capable of processing large volumes yielding amounts of the HMO product of interest that meet, e.g., in the case of a therapeutic compound or composition, the demands for toxicity tests, clinical trials as well as for market supply. In addition to the large volume, a manufacturing scale method, as opposed to simple lab scale methods like shake flask cultivation, is characterized by the use of the technical system of a bioreactor (fermenter) which is equipped with devices for agitation, aeration, nutrient feeding, monitoring and control of process parameters (pH, temperature, dissolved oxygen tension, back pressure, etc.). To a large extent, the behaviour of an expression system in a lab scale method, such as shake flasks, benchtop bioreactors or the deep well format described in the examples of the disclosure, does allow to predict the behaviour of that system in the complex environment of a bioreactor.

With regards to the suitable cell medium used in the fermentation process, there are no limitations. The culture medium may be semi-defined, i.e., containing complex media compounds (e.g., yeast extract, soy peptone, casamino acids, etc.), or it may be chemically defined, without any complex compounds. The carbon-source can be selected from the group consisting of glucose, sucrose, fructose, xylose and glycerol. In one or more exemplary embodiments, the culturing media is supplemented with one or more energy and carbon sources selected form the group containing glycerol, sucrose and glucose. In additional embodiments, lactose is added during the cultivation of the genetically engineered cells as a substrate for the HMO formation.

In one or more exemplary embodiments, the culturing media contains sucrose as the sole carbon and energy source. In one or more exemplary embodiments, the genetically engineered cell comprises one or more heterologous nucleic acid sequence encoding one or more heterologous polypeptide(s) which enables utilization of sucrose as sole carbon and energy source of said genetically engineered cell.

In one or more exemplary embodiments, the genetically engineered cell comprises a PTS-dependent sucrose utilization system, further comprising the scrYA and scrBR operons as described in WO2015/197082 (hereby incorporated by reference).

After carrying out the method of this disclosure, the fucosylated HMO produced can be collected from the cell culture or fermentation broth in a conventional manner.

### Retrieving/Harvesting

The fucosylated human milk oligosaccharide (HMO) is retrieved from the culture medium and/or the genetically engineered cell. In the present context, the term “retrieving” is used interchangeably with the term “harvesting”. Both “retrieving” and “harvesting” in the context relate to collecting the produced HMO(s) from the culture/broth following the termination of fermentation. In one or more exemplary embodiments it may include collecting the HMO(s) included in both the biomass (i.e., the host cells) and cultivation media, i.e., before/without separation of the fermentation broth from the biomass. In other embodiments, the produced HMOs may be collected separately from the biomass and fermentation broth, i.e., after/following the separation of biomass from cultivation media (i.e., fermentation broth).

The separation of cells from the medium can be carried out with any of the methods well known to the skilled person in the art, such as any suitable type of centrifugation or filtration. The separation of cells from the medium can follow immediately after harvesting the fermentation broth or be carried out at a later stage after storing the fermentation broth at appropriate conditions. Recovery of the produced HMO(s) from the remaining biomass (or total fermentation broth) include extraction thereof from the biomass (i.e., the production cells).

After recovery from fermentation, HMO(s) are available for further processing and purification.

The HMOs can be purified according to the procedures known in the art, e.g., such as described in WO2017/152918, WO2017/182965 or WO2015/188834, wherein the latter describes purification of fucosylated HMOs. The purified HMOs can be used as nutraceuticals, pharmaceuticals, or for any other purpose, e.g., for research.

At the end of culturing, the oligosaccharide as product can be accumulated both in the intra-and the extracellular matrix.

The method according to the present disclosure comprises cultivating the genetically engineered microbial cell in a culture medium which is designed to support the growth of microorganisms, and which contains one or more carbohydrate sources or just carbon-source, such as selected from the group consisting of glucose, sucrose, fructose, xylose and glycerol. In one or more exemplary embodiments, the culturing media is supplemented with one or more energy and carbon sources selected form the group containing glycerol, sucrose and glucose.

### Manufactured Product

The term “manufactured product” refers to the one or more HMOs intended as the one or more product HMO(s). Preferably, the product HMOs or composition is produced by a method described herein using a genetically engineered cell described herein.

The data presented in example 1, shows that the α-1,3-fucosyltransferases disclosed herein produces a higher relative amount of 3FL, thus underlining their suitability in large scale manufacturing.

Advantageously, the methods disclosed herein provide an increased overall yield of the product (and/or HMOs in total). This, less by-product formation in relation to product formation, facilitates an elevated product production and increases efficiency of both the production and product recovery process, providing superior manufacturing procedure of HMOs.

The manufactured product may be a powder, a composition, a suspension, or a gel comprising one or more HMOs.

### Sequences

The current application contains a sequence listing in text format and electronical format which is hereby incorporated by reference.

An overview of the SEQ ID NOs used in the present application can be found in table 1 (α-1,3-fucosyltransferase protein sequences) and table 2 (promoter sequences), additional sequences described in the application is amino acid sequence encoding the α-1,3-fucosyltransferase Bgall1 (SEQ ID NO: 5), the DNA sequences encoding the α-1,3-fucosyltransferases (SEQ ID NO: 6-10), the DNA sequence encoding the colanic acid gene cluster from E. coli (SEQ ID NO: 11), the amino acid sequence encoding the Lactose permease from E. coli (SEQ ID NO: 12).

### EXAMPLES

### Methods

Unless stated otherwise, standard techniques, vectors, control sequence elements, and other expression system elements known in the field of molecular biology are used for nucleic acid manipulation, transformation, and expression. Such standard techniques, vectors, and elements can be found, e.g., in: Ausubel et al. (eds.), Current Protocols in Molecular Biology (1995) (John Wiley & Sons); Sambrook, Fritsch, & Maniatis (eds.), Molecular Cloning (1989) (Cold Spring Harbor Laboratory Press, NY); Berger & Kimmel, Methods in Enzymology 152: Guide to Molecular Cloning Techniques (1987) (Academic Press); Bukhari et al. (eds.), DNA Insertion Elements, Plasmids and Episomes (1977) (Cold Spring Harbor Laboratory Press, NY); Miller, J. H. Experiments in molecular genetics (1972.) (Cold spring Harbor Laboratory Press, NY) The embodiments described below are selected to illustrate the disclosure and are not limiting the disclosure in any way.

### Enzymes:

Screening of 50 enzymes with fucosyltransferase activity provided 4 enzymes which showed an α-1,3-fucosyltransferase activity on the glucose moiety on lactose that was better than the recently published enzyme, FutM2 from Bacteroides gallinaceum, which in a fed-batch fermentation demonstrated that a titer of 20.3 g/L 3-FL (Chen et. al., J. Agric. Food Chem. 2022, 70, 1934-1942). The GenBank ID and origin of the 5 fucosyltransferases are provided in table 3.

TABLE 3
List of the enzymes tested in the framework disclosed herein

| Enzyme | GenBank | SEQ | |
|---|---|---|---|
| Name | ID\hat{ } | ID NO | Origin |
| Osc1 | MBC8592593.1 | 1 | Oscillospiraceae bacterium N12 |
| Bbac1 | MBR5851405.1 | 2 | Bacteroidaceae bacterium |
| Murba1 | MBP3303338.1 | 3 | Muribaculaceae bacterium |
| Bacbac1 | MBQ0149235.1 | 4 | Bacteroidales bacterium |
| Bgall1/FutM2* | WP_204430034.1 | 5 | Bacteroides gallinaceum |

\hat{ }the sequences used in the present application may be truncated at the N- or C-terminal as compared to the GenBank sequence these are represented by the SEQ ID NO.
*Bgall1 has been shown to produce 3FL in Chen et. al., J. Agric. Food Chem. 2022, 70, 1934-1942.

### Strains

The strains (genetically engineered cells) constructed in the present application were based on Escherichia coli K-12 DH1 with the genotype: F⁻, λ⁻, gyrA96, recA1, re/A1, endA1, thi-1, hsdR17, supE44. Additional modifications were made to the E. coli K-12 DH1 strain to generate the MDO strain with the following modifications: lacZ: deletion of 1.5 kbp, lacA: deletion of 0.5 kbp, nanKETA: deletion of 3.3 kbp, meIA: deletion of 0.9 kbp, wcaJ: deletion of 0.5 kbp, mdoH: deletion of 0.5 kbp, and insertion of Plac promoter upstream of the gmd gene.

Methods of inserting gene(s) of interest into the genome of E. coli are well known to the person skilled in the art. Insertion of genetic cassettes into the E. coli chromosome can be done using gene gorging (see e.g., Herring and Blattner 2004 J. Bacteriol. 186: 2673-81 and Warming et al 2005 Nucleic Acids Res. 33(4): e36) with specific selection marker genes and screening methods.

Codon optimized DNA sequences encoding individual α-1,3-fucosyltransferases were genomically integrated into the LNnT strain.

The genotypes of the background strain (MDO) and the α-1,3-fucosyltransferase expressing strains capable of producing 3FL are provided in Table 4.

TABLE 4
Genotypes of the strains, capable of producing 3FL, used in the present examples.

| | | α -1,3FT* cDNA |
|---|---|---|
| Strain ref | Genotype | SEQ ID NO |
| MDO | F- λ- ΔendA1 ΔrecA1 ΔrelA1 ΔgyrA96 Δthi-1 glnV44 | — |
| | hsdR17(rK-mK-) ΔlacZ wcaF::Plac ΔnanKETA ΔlacA | |
| | ΔmelA ΔwcaJ ΔmdoH | |
| Osc1 | MDO, Osc1-PglpF, PglpF-CA¹ | 6 |
| Bbac1 | MDO, Bbac1-PglpF, PglpF-CA¹ | 7 |
| Murba1 | MDO, Murba1-PglpF, PglpF-CA¹ | 8 |
| Bacbac1 | MDO, Bacbac1-PglpF, PglpF-CA¹ | 9 |
| Bgall1 | MDO, Bagll1-PglpF, PglpF-CA¹ | 10 |

*1,3FT is an abbreviation of α-1,3-fucosyltransferase, and the DNA sequence is inserted into the genome of the host strain.
¹CA = extra colanic acid gene cluster (gmd-wcaG-wcaH-wcal-manC-manB, SEQ ID NO: 11) under the control of a PglpF promoter at a locus that is different than the native locus.

### Deep Well Assay

Deep Well Assays in the current examples were performed as originally described to Lv et al (Bioprocess Biosyst Eng 20 (2016) 39:1737-1747) and optimized for the purposes of the current disclosure. More specifically, the strains disclosed in the present example were screened in 96 deep well plates using a 4-day protocol. During the first 24 hours, precultures were grown to high densities (OD600 up to 5) and subsequently transferred to a medium that allowed induction of gene expression and product formation.

More specifically, during day 1, fresh precultures were prepared using a basal minimal medium (BMM) (pH 7,0) supplemented with magnesium sulphate (0.12 g/L), thiamine (0.004 g/L) and glucose (5.5 g/L). Basal Minimal medium had the following composition: NaOH (1 g/L), KOH (2.5 g/L), KHzPO4 (7 g/L), NH&HzPO4 (7 g/L), Citric acid (0.5 g/I), trace mineral solution (5 mL/L). The trace mineral stock solution contained; ZnSO~*7H~O 0.82 g/L, Citric acid 20 g/L, MnSO4*H&O 0.98 g/L, FeSO4*7H&O 3.925 g/L, CuSO4*5H~O 0.2 g/L. The pH of the Basal Minimal Medium was adjusted to 7.0 with 5 N NaOH and autoclaved. The precultures were incubated for 24 hours at 34° C. and 1000 rpm shaking and then further transferred to 0.75 mL of a new BMM (pH 7,5) to start the main culture. The new BMM was supplemented with magnesium sulphate (0.12 g/L), thiamine (0.02 g/L), a bolus of glucose solution (0.1-0.15 g/L) and a bolus of lactose solution (5-20 g/L) Moreover, a 20% stock solution of sucrose (40-45 g/L) or maltodextrin (19-20 g/L) was provided as carbon source, accompanied by the addition of a specific hydrolytic enzyme, sucrose hydrolase or glycoamylase, respectively, so that glucose was released at a rate suitable for carbon-limited growth and similar to that of a typical fed-batch fermentation process. The main cultures were incubated for 72 hours at 28° C. and 1000 rpm shaking. For the analysis of total broth, the 96 well plates were boiled at 100° C., subsequently centrifuged, and finally the supernatants were analysed by HPLC.

### Example 1—In Vivo 3FL Synthesis

Genetically modified cells expressing individual α-1,3-fucosyltransferase enzymes were screened for their ability to produce the fucosylated HMO 3FL.

Five enzymes (table 7) were compiled for testing their ability to synthesize 3FL when introduced into a genetically modified cells that produce GDP-Fucose. The enzymes were compared to the known 3FL producing enzyme Bgall1 (Chen et. al., J. Agric. Food Chem. 2022, 70, 1934-1942).

Genetically modified strains expressing the five individual α-1,3-fucosyltransferases (table 3) were generated as described in the “Method” section. The cells were screened in the deep well assay setup as described in the “Method” section.

Table 4 lists the genotype of the strains capable of producing 3FL. The molar content of individual 3FL produced by the strains was measured by HPLC.

The results of the 3FL producing cells are shown in table 5 as the fraction of the total 3FL produced relative to Bgall1 (in percentage, %) produced by each strain.

TABLE 5
Content of individual HMO's as % of total HMO
molar (mM) content produced by each strain relative
to 3FL produced by the Bgall1 strain.

| Enzyme | Relative 3FL production |
|---|---|
| Bbac1 | 121 |
| Bacbac1 | 123 |
| Murba1 | 124 |
| Osc1 | 130 |
| Bgall1 | 100 |

The individual strains only contain a single fucosyltransferase, namely the α-1,3-fucosyltransferase to be tested, and they therefore only produce the HMO 3FL. No α-1,2-fucosyltransferase activity was observed for any of the enzymes.

From the data presented in table 5 it can be seen enzymes Bbac1, Bacbac1, Murba1 and Osc1 all produces a higher amount of 3FL than Bgall1 also known as FutM2, which is described as a high 3FL producer in Chen et. al., J. Agric. Food Chem. 2022, 70, 1934-1942).

1. A genetically engineered cell capable of producing 3FL, comprising a recombinant nucleic acid sequence encoding a fucosyltransferase with α-1,3-fucosyltransferase activity, wherein the fucosyltransferase is selected from the group consisting of,
a. Osc1, comprising the amino acid sequence of SEQ ID NO: 1, or a functional homologue thereof with an amino acid sequence that is at least 90% identical to SEQ ID NO: 1,
b. Bbac1 comprising the amino acid sequence of SEQ ID NO: 2, or a functional homologue thereof with an amino acid sequence that is at least 90% identical to SEQ ID NO: 2,
c. Murba1 comprising the amino acid sequence of SEQ ID NO: 3, or a functional homologue thereof with an amino acid sequence that is at least 90% identical to SEQ ID NO: 3, and
d. Bacbac1 comprising the amino acid sequence of SEQ ID NO: 4, or a functional homologue thereof with an amino acid sequence that is at least 90% identical to SEQ ID NO: 4.

2. The genetically engineered cell according to claim 1, wherein the fucosyltransferase does not have α-1,2-fucosyltransferase activity.

3. The genetically engineered cell according to claim 1, wherein the cell does not produce any DFL.

4. The genetically engineered cell according to claim 1, wherein the recombinant nucleic acid sequence is under the control of one or more promoters, wherein the one or more promoters have a nucleic acid sequence set forth in SED ID NOs: 13-36.

5. The genetically engineered cell according to claim 1, wherein the cell comprises two, three or more genomic copies of the recombinant nucleic acid sequence encoding the fucosyltransferase.

6. The genetically engineered cell according to claim 1, wherein the recombinant nucleic acid sequence encoding a fucosyltransferase with α-1,3-fucosyltransferase activity is expressed from a high copy number plasmid.

7. The genetically engineered cell according to claim 1, wherein the cell further comprises a recombinant nucleic acid sequence encoding a recombinant colanic acid (CA) gene cluster.

8. The genetically engineered cell according to claim 1, wherein said engineered cell is selected from the group consisting of Escherichia Coli, Bacillus subtilis, Lactobacillus lactis, Corynebacterium glutamicum, Yarrowia lipolytica, Pichia pastoris, and Saccharomyces cerevisiae.

9. The genetically engineered cell according to claim 8, wherein the engineered cell is a microorganism is E. coli.

10. A method for producing 3FL, comprising the steps of:
a. cultivating a genetically engineered cell according to claim 1; and
b. culturing the cell according to (a) in a suitable cell culture medium to produce one or more fucosylated HMOs, and
c. optionally, purifying 3FL from the culture medium and/or biomass.

11. The method according to claim 10, wherein the genetically modified cell is cultivated in a suitable culture medium, and in the presence of lactose as the substrate for the α-1,3-fucosyltransferase.

12. (canceled)

13. The genetically engineered cell according to claim 1, wherein the fucosyltransferase is Osc1, comprising the amino acid sequence of SEQ ID NO: 1, or a functional homologue thereof with an amino acid sequence that is at least 90% identical to SEQ ID NO: 1.

14. The genetically engineered cell according to claim 1, wherein the fucosyltransferase is Bbac1 comprising the amino acid sequence of SEQ ID NO: 2, or a functional homologue thereof with an amino acid sequence that is at least 90% identical to SEQ ID NO: 2.

15. The genetically engineered cell according to claim 1, wherein the fucosyltransferase is Murba1 comprising the amino acid sequence of SEQ ID NO: 3, or a functional homologue thereof with an amino acid sequence that is at least 90% identical to SEQ ID NO: 3.

16. The genetically engineered cell according to claim 1, wherein the fucosyltransferase is Bacbac1 comprising the amino acid sequence of SEQ ID NO: 4, or a functional homologue thereof with an amino acid sequence that is at least 90% identical to SEQ ID NO: 4.

17. The genetically engineered cell according to claim 1, wherein the fucosyltransferase is Osc1, consisting of the amino acid sequence of SEQ ID NO: 1, or a functional homologue thereof with an amino acid sequence that is at least 90% identical to SEQ ID NO: 1.

18. The genetically engineered cell according to claim 1, wherein the fucosyltransferase is Bbac1 consisting of the amino acid sequence of SEQ ID NO: 2, or a functional homologue thereof with an amino acid sequence that is at least 90% identical to SEQ ID NO: 2.

19. The genetically engineered cell according to claim 1, wherein the fucosyltransferase is Murba1 consisting of the amino acid sequence of SEQ ID NO: 3, or a functional homologue thereof with an amino acid sequence that is at least 90% identical to SEQ ID NO: 3.

20. The genetically engineered cell according to claim 1, wherein the fucosyltransferase is Bacbac1 consisting of the amino acid sequence of SEQ ID NO: 4, or a functional homologue thereof with an amino acid sequence that is at least 90% identical to SEQ ID NO: 4.
